(19) United States
(12) Patent Application Publication
(10) Pub. No.: US 2021/0001172 A1
(43) Pub. Date: Jan. 7, 2021

(54) Exercise Counting and Form Guidance System

(71) Applicant: Manu Pallatheri Namboodiri, Santa Clara, CA (US)

(72) Inventor: Manu Pallatheri Namboodiri, Santa Clara, CA (US)

(21) Appl. No.: 16/531,084

(22) Filed: Aug. 4, 2019

Related U.S. Application Data
(60) Provisional application No. 62/714,726, filed on Aug. 5, 2018.

(51) Int. Cl. A63B 24/00; G06N 3/08

(52) U.S. Cl. CPC A63B 24/0062; A63B 24/0021; A63B 24/0006; G06N 3/08; A63B 2220/24; A63B 2220/30; A63B 2024/0065; A63B 2220/17; A63B 2024/0056

(57) ABSTRACT

A method and an exercise counting repetition and form guidance system (ECRFGS) for automatically counting multiple repetitions and providing form guidance of an exercise performed by a user in real time are provided. The ECRFGS captures one or more consecutive exercising poses performed by the user, receives the captured consecutive exercising poses, identifies multiple coordinates corresponding to multiple specific body joints of the user , normalizes the captured consecutive exercising poses, analyzes the identified coordinates to determine a plane of orientation, determines one or more relative positions, calculates a distance between at least two of the identified coordinates of the consecutive exercising poses, calculates a maximum angle and a minimum angle between three of the identified coordinates of the consecutive exercising poses, processes the received consecutive exercising poses using the exercise language in the ECRFGS to automatically count the repetitions and form guidance of an exercise performed by the user.

### CROSS-REFERENCE TO RELATED APPLICATIONS

This application claims priority to and the benefit of the provisional patent application titled “Device, Computer Program Product And Method For Counting Exercise Repetitions”, application No. 62/714,726, filed in the United States Patent and Trademark Office on Aug. 5, 2018. The specification of the above referenced patent application is incorporated herein by reference in its entirety.

### BACKGROUND

Physical fitness and physical exercises are correlated with one another. Balanced good health and physical fitness can be achieved by performing physical exercises on a regular basis. The benefits of physical fitness comprise reduced risk of diseases and injuries, and improvement in the quality of life. Some of the exemplary indexes of maintaining the physical fitness for a person are, for example, body flexibility, good immunity, muscular strength, metabolic rate, and body fat ratio. Physical exercises play a significant role in maintaining and improving the physical fitness of a person. The need for a person to maintain good health and desirable physical fitness depends on the performance of regular physical exercises.

The importance of keeping track of the repetitions of the exercises, i.e., the number of times an exercise performed by a user is repeated, and the exercises that are performed using an appropriate form, is to reduce injuries when the exercises are performed and to gain the benefit of the exercises. Examples of regular exercises comprise squat, lunge, plank, pushup, V-up, hip bridge, donkey kicks, jumping jack, mountain climbers, burpees, etc. Examples of weight training exercises comprise bicep curls, shoulder pull downs, and yoga exercises for example, sun salutations, warrior poses, kettlebell exercises, for example, kettlebell swings, etc. Repetition of the exercises performed by a user are usually counted manually using a pen and a paper or using mobile devices. The user must manually remember the number of repetitions of the exercises and input the information into the mobile devices. However, the user tends to lose the rhythm of performing the exercises in all the traditional techniques of counting the repetitions of the exercises. In order to facilitate the counting process of the repetition of the exercises, a stopwatch may be used. However, even with a stopwatch, the user still has to count the repetitions of the exercises, i.e., the number of times the exercises are repeated. Furthermore, a user performing the exercises will be unable to accurately synchronize the start and stop of the repetitions of the exercises with the start and stop of a stopwatch. Therefore, stopwatch and other manually operative solutions for counting the number of repetitions of the exercises of the user are error prone, and therefore do not suit the needs of the user. Furthermore, employing a personal trainer or an expert to be available at all time while performing the exercises to count the repetitions of the exercises, operate the stopwatch, or ensure appropriate exercise form is not feasible.

Conventional methods, systems or devices have been developed to automate an exercise form for monitoring and counting the repetitious movement of the exercises in a live stream or recorded contents, for example, in a pre-captured or real time videos of the user performing the exercises. The conventional methods, systems or devices capture an exercise performed by a user in real time, and the same action is repeated multiple times in consecutive cycles of a relatively uniform length and then counts the number of repetitions of the exercises performed by the user. Moreover, conventional methods, systems, or devices used for counting the number of repetitions of the physical exercises are relatively slow in terms of processing and performance speed because of the complicated or complex algorithm involved for processing the live video streams to count the repetitions of the exercises performed by the user.

Furthermore, conventional systems use at least thirteen to seventeen points for pose estimation. The post estimation techniques use one or more distance capturing cameras, for counting the repetitions of the exercises, and require a person to use wearable sensors to detect the different body movement of the user. The post estimation techniques rely on convolution neural networks (CNN) where the system sequentially analyzes blocks of about twenty non-consecutive image frames per second, and then the cycle length within each block is evaluated using a deep network architecture running on a cloud environment, or a data center of powerful desktop computers to process the results. The processing usually requires the video to stream via high bandwidth connections to high performance computing platforms that lowers the performance of the systems and devices. Thus, the post estimation techniques fail to meet the desired performance, instant feedback, response or speed required for repeated counting of physical exercises, for example, jumping jack, where the counts are to be analyzed and detected in fractions of seconds on a relatively low power and widely available system such as a mobile phone with a normal camera. Moreover, the requirement for high performance, high bandwidth makes real time exercise and motion counting difficult to be monitored on devices and systems that everyone has in their possession, for example, standard mobile phones. An important criteria for designing any system for counting the repetitions of an exercise and provide form guidance of exercises performed by the user is scalability. Scalability ensures that the device works with different types of people, multiple types and varieties of exercises, different powered devices, and with widely available devices.

Hence, there is a long felt but unresolved need for a method and a system for automatically counting the repetitions of an exercise performed by a user in real time and providing immediate form guidance observable as a live video stream over widely available electronic device without additional specialized hardware. Moreover, there is a need for a method and a system that provides scalability for performing multiple and diverse set of exercises with an increased performance level. Furthermore, there is a need for a method and a system that provides instant results on the electronic device without the use of high bandwidth for sending video streams to high performance servers for analysis.

### SUMMARY OF THE INVENTION

This summary is provided to introduce a selection of concepts in a simplified form that are further disclosed in the detailed description of the invention. This summary is not intended to determine the scope of the claimed subject matter.

The method and the system disclosed herein address the above recited need for automatically counting the repetitions of an exercise performed by a user in real time and providing form guidance observable as a live video stream over an electronic device. Furthermore, the method and the system disclosed herein provides scalability for performing multiple and diverse set of exercises with an increased performance level. Furthermore, the method and the system disclosed herein provides near-instant results on the electronic device without the use of high bandwidth for sending video streams to high performance servers for analysis.

The method and the system disclosed herein employs an exercise counting repetition and form guidance system comprising at least one processor configured to execute computer program instructions for automatically counting multiple repetitions and providing form guidance of an exercise performed by a user in real time. The exercise counting repetition and form guidance system captures one or more consecutive exercising poses of the exercise, performed by the user, in real time as a video stream by a capturing module of an electronic device, wherein the captured consecutive exercising poses of the user eliminates the requirement for high bandwidth for streaming the captured consecutive exercising poses of the user for analysis aided by large computing environments. The exercise counting repetition and form guidance system receives the captured consecutive exercising poses in real time, from the capturing module of the electronic device. The exercise counting repetition and form guidance system defines an exercise language comprising a finite set of instructions for automatically counting the repetitions of the exercise and providing form guidance to the user based on the received consecutive exercising poses. The exercise counting repetition and form guidance system identifies multiple coordinates corresponding to multiple specific body joints of the user related to the consecutive exercising poses using the defined exercise language based on the received consecutive exercising poses of the user, wherein identifying the coordinates of the consecutive exercising poses of the user comprises using a convolutional neural network (CNN) and deep learning algorithms for detecting image and video objects, human pose estimation, and computer vision analysis. The defined exercise language in the exercise counting repetition and form guidance system automatically counts the repetitions and provides form guidance for any type of exercise without changing the deep learning methods and the codes, for example, the finite set of instructions, used in the exercise counting repetition and form guidance system. The defined exercise language in the exercise counting repetition and form also increases scalability. Therefore, the defined exercise language in the exercise counting repetition and form guidance system automatically counts the repetitions and provides form guidance for a plurality of exercises using the same deep learning methods and the finite set of instructions used in the exercise counting repetition and form guidance system.

The exercise counting repetition and form guidance system normalizes the captured consecutive exercising poses of the user based on the identified coordinates related to the consecutive exercising poses, wherein the normalization of the consecutive exercising poses of the user comprises mapping a height of the captured consecutive exercising poses of the user to a predefined standard height of the user, and selectively mapping the captured consecutive exercising poses of the user to their corresponding rotational equivalents. The exercise counting repetition and form guidance system analyzes the identified coordinates associated with the normalized consecutive exercising poses of the user for determining a plane of orientation of the user. The exercise counting repetition and form guidance system determines one or more relative positions of the user based on the determination of the plane of orientation of the consecutive exercising poses of the user, wherein the relative positions corresponds to a position between and among the identified coordinates of the consecutive exercising poses of the user. The exercise counting repetition and form guidance system calculates a distance between at least two of the identified coordinates of the consecutive exercising poses of the user based on the determined plane of orientation and the determined one or more relative positions of the identified coordinates of the consecutive exercising poses of the user as a percentage of the predefined height.

The exercise counting repetition and form guidance system calculates a maximum angle and a minimum angle between three of the identified coordinates of the consecutive exercising poses of the user based on the calculated distance between at least two of the identified coordinates of the consecutive exercising poses of the user. Determining the plane of orientation of the user, the relative positions of the identified coordinates of the consecutive exercising poses of the user, the angles among the identified coordinates of the consecutive exercising poses of the user, and calculating the distance between at least two of the identified coordinates of the consecutive exercising poses of the user as a percentage of the predefined height further comprise a predefined confidence interval to determine an acceptable error range.

The exercise counting repetition and form guidance system processes the received consecutive exercising poses of the user by utilizing one or more stored instructions in the exercise counting repetition and form guidance system from the exercise language based on the calculated maximum angle and the minimum angle between three of the identified coordinates of the consecutive exercising poses of the user, thereby automatically counting the repetitions of an exercise performed by the user and providing form guidance by ensuring the angles are within acceptable parameters. The exercise counting repetition and form guidance system further processes higher image frames per second of the captured consecutive exercising poses of the user thereby performing fast moving exercises. The exercise counting repetition and form guidance system provides appropriate exercise form guidance to the user who is performing the exercise in real time, based on the processed consecutive exercising poses of the user, by verifying the exercise being performed by the user is in the appropriate form.

The exercise counting repetition and form guidance system further comprises determining one or more calorie counts of the user, the efforts and intensity of the user during exercise routines, the speed of performing the exercise by the user, and the halt times based on the automatic counting of the repetitions of the exercise and the time elapsed performing the exercise, when the exercise is being done by the user, and providing instant feedback and appropriate exercise form guidance to the user. The exercise counting repetition and form guidance system for automatically counting the repetitions and ensuring form of the exercise further comprises information of one or more predefined exercises performed in sequence by the user. The exercise counting repetition and form guidance system determines the plane of orientation of the consecutive exercising poses for the predefined exercises performed in sequence by the user corresponds to specific detected points of the user, wherein the specific detected points of the user comprises horizontal, vertical, and angular plane to determine the plane of orientation of the user, and enables to provide form guidance in an appropriate form.

The relative positions of the user for performing the predefined exercises comprises in front of, behind, above, below, the identified coordinates of the consecutive exercising poses of the user. The exercise counting repetition and form guidance system processes the received consecutive exercising poses of the user for predefined exercises further comprises determining an end of a predefined exercise pose performed by the user.

In one or more embodiments, related systems comprise circuitry and/or programming for effecting the methods disclosed herein; the circuitry and/or programming can be any combination of hardware, software, and/or firmware configured to effect the methods disclosed herein depending upon the design choices of a system designer. Also, in an embodiment, various structural elements can be employed depending on the design choices of the system designer.

### BRIEF DESCRIPTION OF THE DRAWINGS

The foregoing summary, as well as the following detailed description of the invention, is better understood when read in conjunction with the appended drawings. For illustrating the invention, exemplary constructions of the invention are shown in the drawings. However, the invention is not limited to the specific methods and components disclosed herein. The description of a method step or a component referenced by a numeral in a drawing is applicable to the description of that method step or component shown by that same numeral in any subsequent drawing herein.

FIGS. 1A-1B illustrate a method for automatically counting multiple repetitions and providing form guidance of an exercise performed by a user in real time.

FIG. 2A-2C exemplarily illustrates flow charts for automatically counting multiple repetitions and providing form guidance of an exercise performed by a user in real time.

FIG. 3 exemplarily illustrates an embodiment of the exercise repetition and form guidance system where the user is in the standing position with different body joints.

FIGS. 4A-4H exemplarily illustrates an embodiment of the exercise repetition and form guidance system where the user performs a jumping jack exercise.

FIGS. 5A-5C exemplarily illustrates an embodiment of the exercise repetition and form guidance system where the user performs a burpee exercise.

FIG. 6 exemplarily illustrates an embodiment of the exercise repetition and form guidance system where the user performs a mountain climber exercise.

FIG. 7 exemplarily illustrates an embodiment of the exercise repetition and form guidance system where the user performs exercises with pushups.

FIG. 8 exemplarily illustrates an embodiment of the exercise repetition and form guidance system where the user performs an exercise of kettle bell swing.

FIG. 9 exemplarily illustrates an embodiment of the exercise repetition and form guidance system where the user performs a bridge exercise.

FIG. 10 exemplarily illustrates an embodiment of the exercise repetition and form guidance system where the user performs weightlifting exercise.

FIG. 11 exemplarily illustrates an architectural diagram of a system comprising an exercise counting repetition and form guidance system for automatically counting multiple repetitions and providing form guidance of an exercise performed by a user in real time.

### DETAILED DESCRIPTION OF THE INVENTION

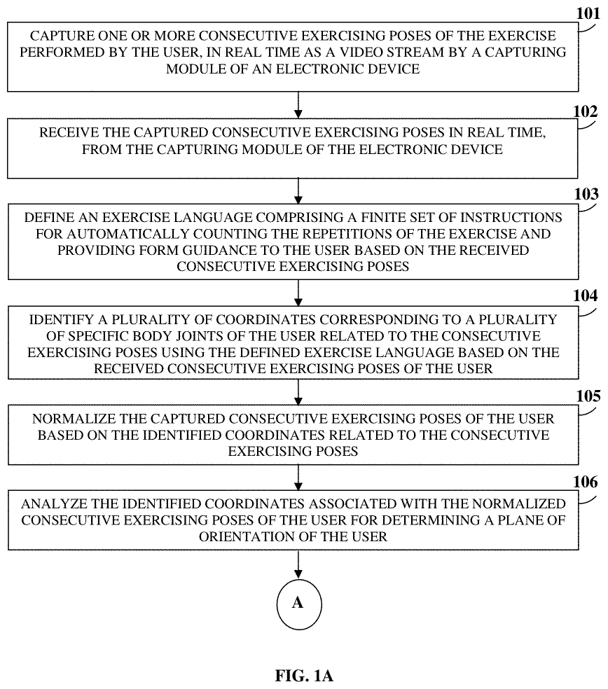

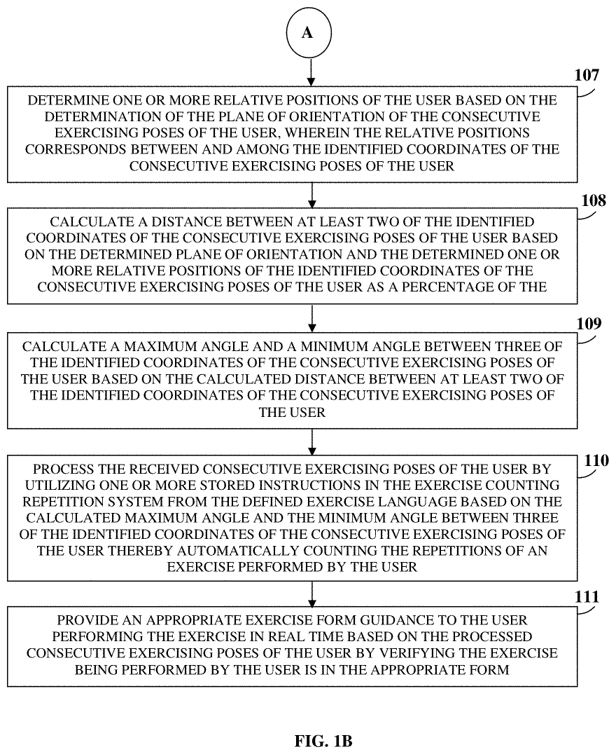

FIGS. 1A-1B illustrate a method for automatically counting multiple repetitions and providing form guidance of an exercise performed by a user in real time. The method disclosed herein is implemented on an electronic device. The electronic device employs an exercise counting repetition and form guidance system comprising a non-transitory computer readable storage medium to store computer program instructions defined by the exercise counting repetition and form guidance system and at least one processor communicatively coupled to the non-transitory computer readable storage medium. The at least one processor is configured to execute the defined computer program instructions for automatically counting multiple repetitions and providing form guidance of an exercise performed by a user in real time. The electronic device, is for example, a mobile phone, smartphone, personal digital assistant, wearable computing device such as the Google Glass® of Google Inc., Apple Watch® of Apple Inc., etc., touch centric devices, portable electronic devices, network enabled computing devices, interactive network enabled communication devices, etc.

The exercise counting repetition and form guidance system captures 101 one or more consecutive exercising poses of the exercise, performed by the user, in real time as a video stream by a capturing module of an electronic device. The capturing module is, for example, a video camera of the electronic device. When the user starts performing any exercise, the user triggers the exercise counting repetition and form guidance system deployed on the electronic device. The exercise counting repetition and form guidance system allows the user to select any specific exercise to be monitored. The user selects the exercise to be monitored by himself, or the user assists other users in monitoring the exercises performed by them. The video camera on the electronic device captures the live video of the exercise being performed by the user. The analysis of captured consecutive exercising poses of the user on the device itself eliminates the requirement for a high bandwidth connection, for example, an internet connection, wireless connection, etc., for streaming the captured consecutive exercising poses of the user for analysis aided by large computing environments. The exercise counting repetition and form guidance system further processes a higher number of image frames per second of the captured consecutive exercising poses of the user thereby performing fast moving exercises.

Artificial intelligence (AI) pose estimation models are used in understanding the human body. The artificial intelligence (AI) pose estimation models retrieve 13-17 body joints related to consecutive exercising poses of the user. When each image frame in the number of image frames captured related to consecutive exercising poses of the user is processed, each body joint corresponds to a body joint, or landmark on the body of the user, for example, left shoulder, left eye, etc. If the computational power of the electronic device is high, the exercise counting repetition and form guidance system utilizes all the 17 body joints of the user, and if the computational power of the electronic device is less, the exercise counting repetition and form guidance system utilizes less than 17 body joints. In an embodiment, if the computational power of the electronic device is less, the exercise counting repetition and form guidance system utilizes only 5 body joints of the user thereby reducing the accuracy, but still enables the electronic device to function at high speed. Since, the exercise counting repetition and form guidance system utilizes only 5 body joints or less, that reduces the computation requirements during the analysis and provides high speed accuracy to automatically count the multiple repetitions of an exercise performed by the user. Therefore, the exercise counting repetition and form guidance system processes more image frames per second, compared to a model that requires the complete 13-17 body joints of the user.

The exercise counting repetition and form guidance system receives 102 the captured consecutive exercising poses in real time, from the capturing module of the electronic device. The capturing module of the electronic device continuously tracks the user performing an exercise. The exercise counting repetition and form guidance system defines 103 an exercise language comprising a finite set of instructions for automatically counting the repetitions of the exercise and providing form guidance to the user based on the received consecutive exercising poses of the user. The exercise counting repetition and form guidance system identifies 104 multiple coordinates corresponding to multiple specific body joints of the user related to the consecutive exercising poses using the defined exercise language based on the received consecutive exercising poses of the user. The step of identifying the coordinates of the consecutive exercising poses of the user comprises using a convolutional neural network (CNN) and deep learning algorithms by the exercise counting repetition and form guidance system for detecting image and video objects, human pose estimation, and computer vision analysis. The deep learning method used is a computer vision technique referred as pose estimation. The exercise counting repetition and form guidance system identifies the specific body joints and points of interest on a human body and returns a specific X, Y coordinates of those body joints, for example, left shoulder, right ankle, left eye, etc., based on an image or a frame from a video stream captured from the capturing module of the electronic device. The defined exercise language in the exercise counting repetition and form guidance system automatically counts the repetitions and provides form guidance for any type of exercise without changing the deep learning methods and the codes, for example, the finite set of instructions, used in the exercise counting repetition and form guidance system and also increases the scalability of the exercise counting repetition and form guidance system. The defined exercise language in the exercise counting repetition and form guidance system automatically counts the repetitions and provides form guidance for a plurality of exercises using the deep learning methods and the finite set of instructions used in the exercise counting repetition and form guidance system.

In an embodiment, the coordinates correspond to multiple groups of points or coordinates on the body of the user, based on the specific type of exercise. In another embodiment, the coordinates correspond to five or less coordinates thereby increasing the analysis of image frames captured by the capturing module of the electronic device. The multiple groups of points or coordinates on the body of the user comprise, for example, a head, two wrists, and two feet of a user corresponding to five points or coordinates on the body of the user, a head, two wrists, and hip of a user corresponding to four points or coordinates on the body of the user, a head, two wrists of a user corresponding to three points or coordinates on the body of the user, a head and two feet of a user corresponding to three points or coordinates on the body of the user, two wrists and hip of a user corresponding to three points or coordinates on the body of the user, one wrist and head of a user corresponding to two points or coordinates on the body of the user, and one wrist corresponding to a single point or coordinate on the body of the user.

The exercise counting repetition and form guidance system normalizes 105 the captured consecutive exercising poses of the user based on the identified coordinates related to the consecutive exercising poses. In an embodiment, the normalization of the consecutive exercising poses of the user comprises mapping a height of the captured consecutive exercising poses of the user to a predefined standard height of the user, and selectively mapping the captured consecutive exercising poses of the user to their corresponding rotational equivalents. The predefined standard height is, for example, six feet or 180 centimeters. In another embodiment, the rotational equivalents correspond to capturing exercising poses that are viewed from angle 0-90 degrees. The captured exercising poses are viewed from the front face of the user or the side face of the user or at a certain angle greater than 0 degrees and less than 90 degrees, for example, around 45 degrees facing the user. The captured exercising poses are then mathematically rotated to the corresponding coordinates related to the consecutive exercising poses based on the received consecutive exercising poses of the user to either front facing of the user, for example, 0 degrees or side facing of the user, for example, 90 degrees depending on obtaining the clearest view of the specific exercise performed by the user. The step of normalization is a pre-condition for automatically counting multiple repetitions and providing form guidance of an exercise and the normalization is performed for each captured consecutive exercising poses relating to the exercise being performed by the user.

The exercise counting repetition and form guidance system analyzes 106 the identified coordinates associated with the normalized consecutive exercising poses of the user for determining a plane of orientation of the user. In an embodiment, the identified coordinates related to the consecutive exercising poses range from, for example 13-17 or fewer, for example, 5 or less if the exercise counting repetition and form guidance system is deployed on a low performance electronic device corresponding to multiple specific body joints of the user related to the consecutive exercising poses is derived using the human pose estimation deep learning artificial intelligence (AI) model. The current exercising pose of the user is determined using two or more X coordinates and Y coordinates from the set of derived coordinates of the consecutive exercising poses of the user and compared and analyzed to determine the plane of orientation of those coordinates in a specific captured exercising pose of the user.

The exercise counting repetition and form guidance system determines one or more relative positions 107 of the user based on the determination of the plane of orientation of the consecutive exercising poses of the user, wherein the relative positions corresponds to a position between and among the identified coordinates of the consecutive exercising poses of the user. The identified coordinates of the consecutive exercising poses of the user are, for example, head of the user, hip of the user, wrists of the user, and feet of the user. The relative positions of one of the coordinates is, for example, above or below or front or behind the other referenced coordinate of the consecutive exercising poses of the user and can be used to identify the captured pose of the user.

The exercise counting repetition and form guidance system calculates a distance 108 between at least two of the identified coordinates of the consecutive exercising poses of the user based on the determined plane of orientation and the determined one or more relative positions of the identified coordinates of the consecutive exercising poses of the user as a percentage of the predefined height. In an embodiment, two of the identified coordinates of the consecutive exercising poses of the user corresponds to the head of the user, hip of the user, and feet of the user. In another embodiment, the exercise counting repetition and form guidance system calculates a distance from the specific identified coordinates of the consecutive exercising poses of the user to a fixed position, for example, a floor on which the user is standing as a percentage of the predefined height. The mathematical formulae for calculating the distance between at least two of the identified coordinates of the consecutive exercising poses of the user based on the determined plane of orientation and the determined one or more relative positions of the identified coordinates of the consecutive exercising poses of the user as a percentage of the predefined height is represented as: percentage of the predefined height=d/H; where ‘d’ is the distance between two of the five identified coordinates, for example, knee and wrist of the user, and ‘H’ is the distance between the feet of the user and head of the user.

The exercise counting repetition and form guidance system calculates a maximum angle and a minimum angle 109 between three of the identified coordinates of the consecutive exercising poses of the user based on the calculated distance between at least two of the identified coordinates of the consecutive exercising poses of the user. The maximum angle between three of the identified coordinates of the consecutive exercising poses of the user is, for example, between the ankle of the user, knee of the user, and waist of the user. The minimum angle between three of the identified coordinates of the consecutive exercising poses of the user is, for example, between the ankle of the user, knee of the user, and waist of the user.

The exercise counting repetition and form guidance system processes 110 the received consecutive exercising poses of the user by utilizing one or more stored instructions in the exercise counting repetition and form guidance system from the defined exercise language based on the calculated maximum angle and the minimum angle between three of the identified coordinates of the consecutive exercising poses of the user thereby automatically counting the repetitions of an exercise performed by the user. The one or more stored instructions in the exercise counting repetition and form guidance system are defined by the exercise language. As used herein, “exercise language” refers to a finite or limited set of alphabets or instructions stored in the exercise counting repetition and form guidance system that can be combined to describe any type of exercise and enables counting multiple repetitions of the exercise performed by the user and provides form guidance to the user. The exercise language is a finite instruction set stored in the exercise counting repetition and form guidance system. The exercise counting repetition and form guidance system combines one or more of these finite set of instructions in distinct combinations that is sufficient to automatically count multiple repetitions of an exercise performed by the user and provide basic exercise form guidance for different types of exercises performed by the user. By using a limited, finite and known set of functions, the exercise language allows for separation of the deep learning model from the exercises themselves, thus enabling future scalability and even adoption of exercises not known at the present time. The exercise language set of finite instructions enables actual counting of multiple repetitions of the exercise performed by the user and provides form guidance to the user. In an embodiment, one or more exercise language steps or instructions are performed depending on the specific type of exercise to understand the specific captured consecutive exercising poses of the user to determine an increase in the count of the multiple repetitions of the exercise and to guide the exercise with appropriate form performed by the user. The exercise counting repetition and form guidance system provides 111 appropriate exercise form guidance to the user performing the exercise in real time based on the processed consecutive exercising poses of the user by verifying the exercise being performed by the user is in the appropriate form.

The exercise counting repetition and form guidance system processes the received consecutive exercising poses of the user in a specific sequence. The exercise counting repetition and form guidance system implements or executes one or more steps or instructions from the exercise language in any order depending on the specific exercise performed by the user to determine the consecutive exercising poses, provide an appropriate form guidance within that consecutive exercising pose, detects the correct consecutive exercising pose of the user, and increments the count of the consecutive exercising poses of the user. In an embodiment, Instruction 1) AND Instruction 2 OR Instruction 1 AND Instruction3 defines ExerciseA performed by the user. The method disclosed herein requires a few select instructions from the exercise language for automatically counting multiple repetitions of the exercise performed by the user and providing form guidance to the user based on the nature of the selected exercise by the user.

The exercise counting repetition and form guidance system enables the automatic counting of repetition of the exercise and provides form guidance using different artificial intelligence (AI) pose estimation models depending on the performance requirements on the electronic device. The pose estimation techniques comprise, five-point pose estimation, for example, feet, wrists, head of the user for fast moving exercises, for example, jumping jacks, high knees, etc., optionally a second five point pose estimation, for example, waist, knees, and head of the user for fast moving lower body exercises, a complete thirteen point pose estimation for performing slow moving exercises to count the repetitions of the exercises and providing form guidance to the user. All these pose estimation techniques accounts for the performance limitations on the electronic device and counting the repetitions of the exercises in real time.

The following functions represent the core and finite set of exercise language instructions and contributes an exercise markup language for automatically counting the repetitions and providing form guidance of the exercises performed by the user in real time.
NormalizePose (Front/Side):
   The above instruction returns the pose of the user as six feet and rotates the points, so the view of the user is sideways or front for calculating the angle between the identified coordinates of the user. The instruction is used if the camera is not capturing the pose of the user straight on or sideways for specific exercises.
UserHeight ( )
   The above instruction calculates and stores the height of the user for the functions described below. Every distance or confidence calculation described below is represented as a percentage of this height of the user.
DistanceBetweenJoints([greaterthan/lessthan/equal], Coord1, Coord2, confidencelnterval)—
   The above instruction returns yes/no responses between two coordinates of the user as percent of 6 feet height (yes, no) responses.
IsSamePlane([horizontal/vertical/specificangle], coordList1, coorList2, coordList3, confidencelnterval)—
   The above instruction returns yes/no responses if the coordinates are on the same plane, that is if similar x or y axis for coordinates in the parameters. The confidence interval is a percent of 6 feet height of the user.
IsRelativePosition ([Above/below/front/behind], coordList1, coordList2, confidencelnterval)—
   The above instruction returns a Yes/No—the first list of coordinates in the requested relative position (above, below, front or back) of the second list of coordinates?
IsMaxAngle (coordList1, coordList2, coordlist3, [greaterthan/lessthan/equal/between],angle1, angle2)
   The above instruction returns “Is the max angle between the three coordinates greaterthan/lessthan/equal angle1, or betweenangle1 and angle2. The middle joint is the middle of the angle.
IsMinAngle (coordList1, coordList2, coordlist3, [greatterthan/lessthan/equal/between],angle1, angle2)
   The above instruction returns “Is the min angle between the three coordinates greaterthan/lessthan/equalangle1, or between angle1 and angle2

The exercise counting repetition and form guidance system further comprises determining one or more calorie counts of the user, the efforts and intensity of the user during exercise routines, the speed of performing the exercise by the user, and the halt times based on the automatic counting of the repetitions of the exercise and the time elapsed performing the exercise, when the exercise is being done by the user and providing instant feedback and appropriate exercise form guidance to the user. The exercise counting repetition and form guidance system determines the plane of orientation of the user, determines the relative positions of the identified coordinates of the consecutive exercising poses of the user, calculates the angles among the identified coordinates of the consecutive exercising poses of the user, and calculates the distance between at least two of the identified coordinates of the consecutive exercising poses of the user as a percentage of the predefined height further comprising a predefined confidence interval to determine an acceptable error range. The confidence interval value is, for example, fixed by the vendor considering the practical scenarios of accuracy, noise, and artificial intelligence (AI) model capability.

The confidence interval is used as a buffer during the calculations to account for slight inaccuracies in how the deep learning methods used by the exercise counting repetition system returns the coordinates for each specific body joints or landmark of the user. For example, if the user performs a jumping jack exercise, the exercise counting repetition system increments the count of the jumping jack exercise when the wrists of the user are above the head of the user. However, assume there is a slight error in the calculation of the coordinates of the consecutive exercising poses of the user. The confidence interval of, for example, 10%, enables the count increment to proceed even if the wrist of the user is not completely above the head of the user but within 10% of error range. The confidence interval defines the range of error as the larger the confidence interval, the larger is the allowed error in the calculation of the count of the exercise and vice versa.

The exercise counting repetition and form guidance system provides instant feedback based on the completion of the repetition of the exercises performed by the user and indicates if the exercise is performed using appropriate form guidance. For example, if the user performs pushups exercise, and the user is supposed to perform the pushups until a count of 15 pushups exercise. If the user reaches the count of 12 pushups exercise, the exercise counting repetition system records the 12 pushups exercise performed by the user and encourages the user to push harder in order to complete the target list of 15 pushups exercise. The exercise counting repetition and form guidance system sends a signal to the user that reads, for example, “Push on, only 3 more to go”. Furthermore, the exercise counting repetition and form guidance system verifies that the user performs the pushup exercise with good form, for example, the waist of the user should not be too low while performing the exercise. While the pushup is being performed by the user, the exercise counting repetition and form guidance system recognizes a low waist of the user and guides the user in real time instantly by providing a message that reads, for example, “Your waist is too low. Please hold it up when you are doing the pushup”. The instant feedback provided by the exercise counting repetition and form guidance system is important to ensure that the user performs the exercise correctly and thus the user does not risk injury and receive the benefits of the exercise.

In an embodiment, the exercise counting repetition and form guidance system for automatically counting the repetitions of the exercise further comprises information of one or more predefined exercises performed in sequence by the user. The exercise counting repetition and form guidance system determines the plane of orientation of the consecutive exercising poses for the predefined exercises performed in sequence by the user corresponds to specific detected points of the user. The specific detected points of the user comprise horizontal, vertical, and angular plane to determine the plane of orientation of the user. The relative positions of the user for performing the predefined exercises comprises in front of, behind, above, and below the identified coordinates of the consecutive exercising poses of the user. The exercise counting repetition and form guidance system for processing the received consecutive exercising poses of the user for predefined exercises further comprises determining an end of a predefined exercise pose performed by the user.

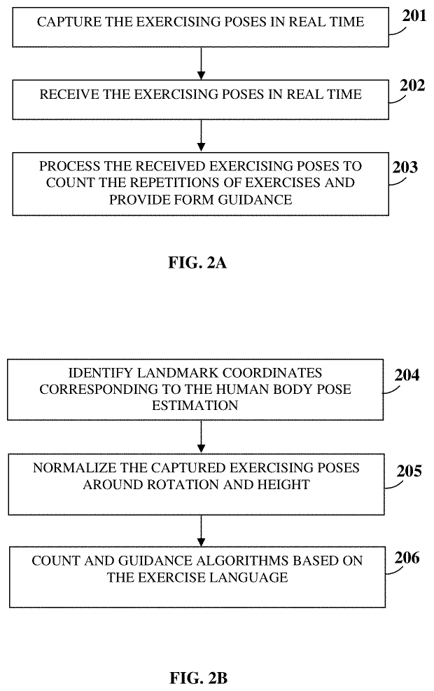

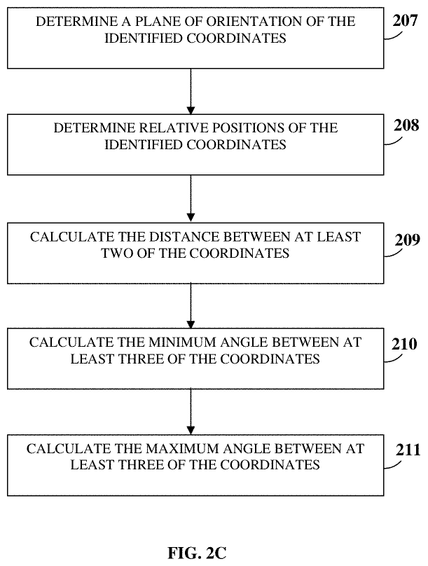

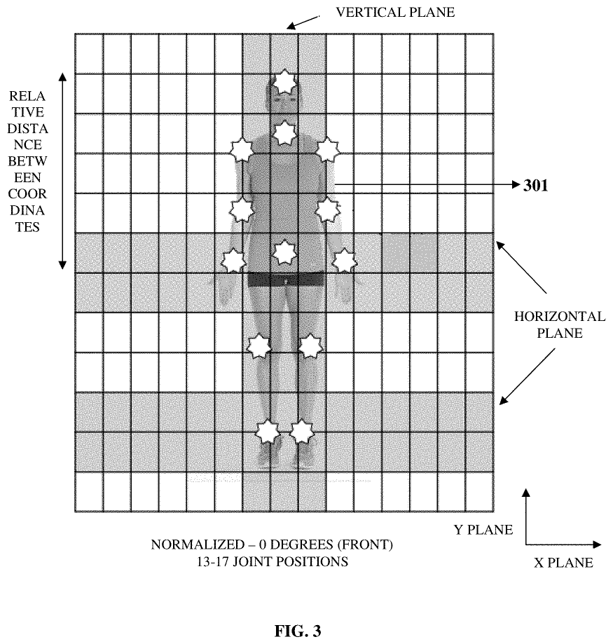

FIGS. 2A-2C exemplarily illustrates flow charts for automatically counting multiple repetitions and providing form guidance of an exercise performed by a user in real time. FIG. 2A exemplarily illustrates a flowchart where the exercise counting repetition and form guidance system captures 201 the consecutive exercising poses performed by the user in real time, receives 202 the captured exercising poses from the capturing module of the electronic device, and processes 203 the received consecutive exercising poses to count the repetitions of the exercise and provides form guidance as exemplarily illustrated in the detailed description of FIGS. 1A-1B. FIG. 2B exemplarily illustrates a flowchart where the exercise counting repetition and form guidance system identifies 204 the body joint or landmark coordinates corresponding to the human body pose estimation, normalizes 205 the captured consecutive exercising poses around the rotation and height of the user, and counts 206 the repetitions of the exercise and provides form guidance based on the exercise language as exemplarily illustrated in the detailed description of FIGS. 1A-1B. FIG. 2C exemplarily illustrates a flowchart where the exercise counting repetition and form guidance system determines 207 a plane of orientation of the identified coordinates of the consecutive exercising poses, determines 208 one or more relative positions of the identified coordinates of the user, calculates 209 the distance between at least two of the coordinates, calculates the minimum angle 210 between at least three of the identified coordinates, and calculates the maximum angle 211 between at least three of the identified coordinates of the user as exemplarily illustrated in the detailed description of FIGS. 1A-1B.

FIG. 3 exemplarily illustrates an embodiment of the exercise repetition and form guidance system where the user 301 is in the standing position with different body joints. The exercise counting repetition and form guidance system identifies multiple coordinates corresponding to multiple specific body joints related to the consecutive exercising poses of the user 301. FIG. 3 exemplarily illustrates the vertical plane and the horizontal plane with the user 301 in the standing position. In an embodiment, the user 301 is in the standing position with at least 13-17 identified coordinates corresponding to specific body joints of the user 301. The captured exercising poses are rotated to the corresponding coordinates related to the consecutive exercising poses based on the received consecutive exercising poses of the user to front facing of the user 301, for example, 0 degrees. The exercise counting repetition and form guidance system normalizes the captured consecutive exercising poses of the user 301 and calculates the relative distance between the identified coordinates of the consecutive exercising poses of the user 301.

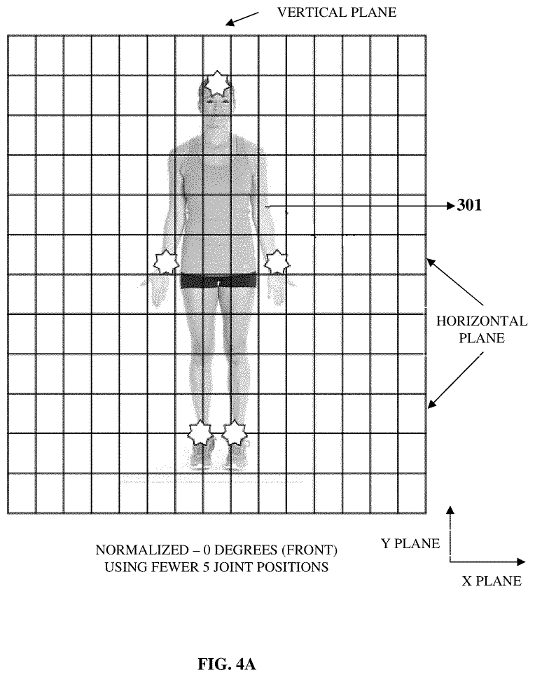

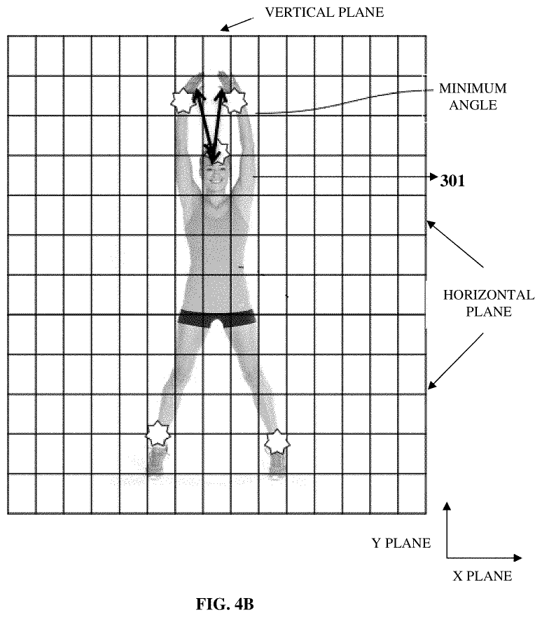

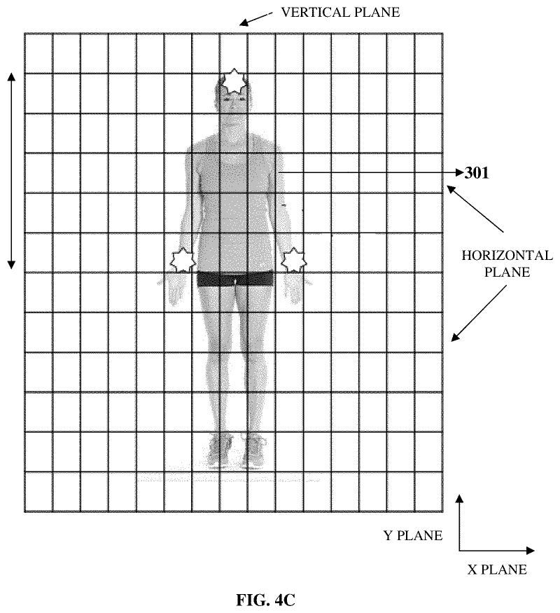

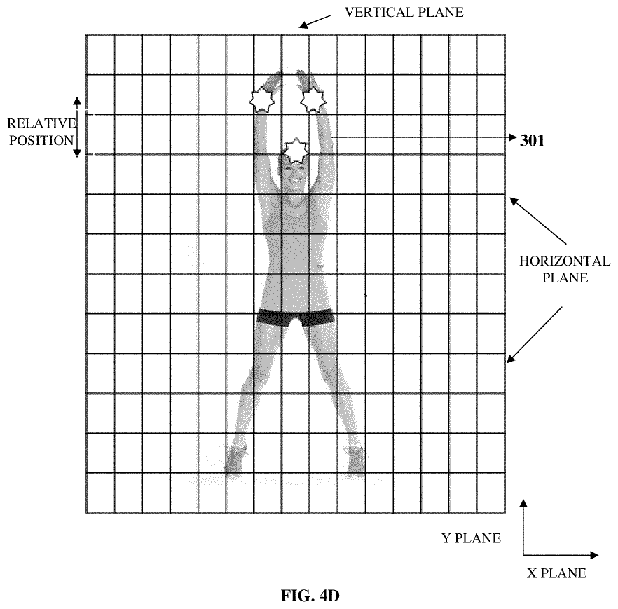

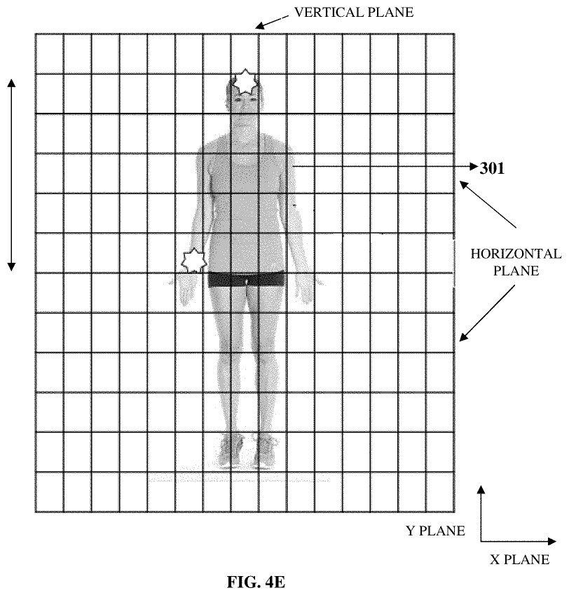

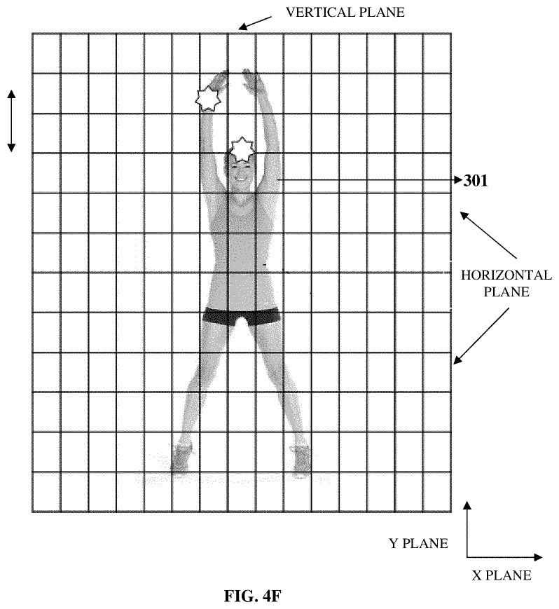

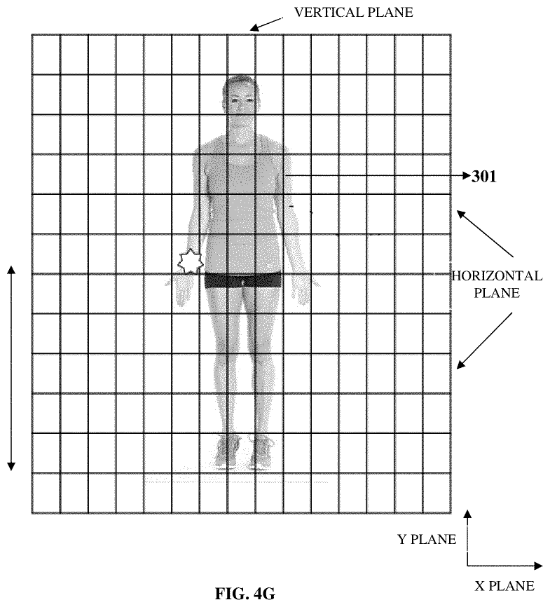

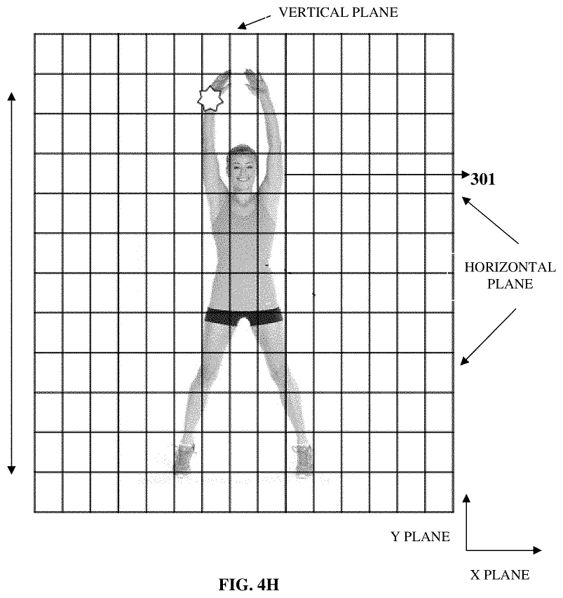

FIGS. 4A-4H exemplarily illustrates an embodiment of the exercise repetition and form guidance system where the user 301 performs a jumping jack exercise. The exercise counting repetition and form guidance system continuously receives the captured consecutive exercising poses in real time from the capturing module of the electronic device. The exercise counting repetition and form guidance system continuously tracks the user 301 performing the jumping jack exercise and identifies the current two poses of the exercise performed by the user 301. The exercise counting repetition and form guidance system processes the received consecutive exercising poses of the user 301 using deep learning methods to derive a set of coordinates on the X-Y coordinate planes as exemplarily illustrated in FIG. 4A.

FIGS. 4A, 4C, 4E, and 4G exemplarily illustrate the user 301 in a standing pose to perform the specified exercise. FIGS. 4B, 4D, 4F, and 4H exemplarily illustrate the user 301 in a jump pose to perform the specified exercise. The exercise counting repetition and form guidance system deployed on the electronic device normalizes the captured consecutive exercising poses of the user 301 based on the identified coordinates related to the consecutive exercising poses of the user 301. In an embodiment, the received consecutive exercising poses from the capturing module are, for example, a standing pose of the user 301 or a jump pose of the user 301 depending on the video frame at that particular instant. The normalization of the captured standing pose of the user 301, or jump pose of the user 301 comprises mapping the height of the captured standing pose of the user 301 or the jump pose of the user 301 to a predefined standard height, for example, 6 feet or 180 centimeters and selectively mapping the captured standing pose of the user 301 or the jump pose of the user 301 to their corresponding rotational equivalents. The rotational equivalents correspond to the captured consecutive exercising poses of the user 301 viewed from an angle of 0 degrees, that is, viewed from the front face of the user 301, as the user 301 is always in the standing position while performing the exercise.

Following the process of normalization, the exercise counting repetition and form guidance system determines one or more relative positions of the user 301. For a specific jumping jack exercise, the exercise counting repetition and form guidance system determines the relative positions of the two wrists coordinates of the user 301 with respect to the head coordinates of the user 301. The determination of the relative positions of the two wrists coordinates of the user 301 with respect to the head coordinates of the user 301 comprises a confidence interval, for example, 10%. If the user 301 is in the standing pose as exemplarily illustrated in FIG. 4A, the relative positions of the coordinates of the two wrists of the user 301 are identified below the head of the user 301 and the confidence interval of 10% is taken into account for further calculations by the exercise counting repetition and form guidance system. The exercise counting repetition and form guidance system after detecting the standing pose of the user 301, receives the next pose in sequence, that is, the jump pose of the user 301. If the user 301 is in the jump pose as exemplarily illustrated in FIG. 4B, the relative positions of the coordinates of the two wrists of the user 301 are identified above the head of the user 301 and the confidence interval of 10% is taken into account for further calculations to increase the counter value of the repetition of the exercise by the exercise counting repetition and form guidance system. The exercise counting repetition and form guidance system provides appropriate pose guidance to the user 301 to ensure that the hands of the user 301 are close together over the head of the user 301. The pose guidance is provided by adding further instructions from the exercise language of the exercise counting repetition system. The minimum angle among the two wrists of the user 301 and the head of the user 301 is smaller than 40 degrees as exemplarily illustrated in FIG.4B.

For many exercises, there is a possibility of reducing the specific detection points of the user 301 that are needed to count the repetitions of the exercise accounting for performance constrained devices. In an embodiment, for the jumping jack exercise performed by the user 301, the exercise counting repetition and form guidance system identifies only three coordinates of the consecutive exercising poses of the user 301, for example, one for the head of the user 301, and two for the wrists of the user 301 as exemplarily illustrated in FIGS. 4C-4D to count the repetitions of the exercise by analyzing the two poses forming the jumping jack exercise. In another embodiment, for the jumping jack exercise performed by the user 301, the exercise counting repetition and form guidance system identifies only two coordinates of the consecutive exercising poses of the user 301, for example, one for the head of the user 301, and any one of the two wrists of the user 301 as exemplarily illustrated in FIGS. 4E-4F to count the repetitions of the exercise by analyzing the two poses forming the jumping jack exercise. In another embodiment, for the jumping jack exercise performed by the user 301, the exercise counting repetition and form guidance system identifies only one coordinate of the consecutive exercising poses of the user 301, for example, only one of the two wrists of the user 301 as exemplarily illustrated in FIGS. 4G-4H to count the repetitions of the exercise by analyzing the two poses forming the jumping jack exercise. The exercise counting repetition and form guidance system analyzes the relative distance from the wrist of the user 301 to the floor for each pose of the user 301 and calculates whether the hand of the user 301 is positioned high or low while performing the jumping jack exercise. In some of the exercises, there is a possibility of lower accuracy with fewer coordinates while in certain exercises all of the five coordinates are not necessary for accuracy.

The pseudo code below shows how the exercise language and the finite set of instructions and functions can count the repetitions of the exercises and provide form guidance to the user 301 if the user 301 performs the jumping jack exercise. The exercise counting repetition and form guidance system normalizes the captured exercising poses of the user 301, for example, front pose of the user 301. The exercise counting repetition and form guidance system identifies two of the received consecutive exercising poses of the user 301. If the user 301 is in the standing pose, the exercise counting repetition and form guidance system determines the plane of orientation of the user 301, calculates the distance between the identified coordinates corresponding to specific body joints of the user 301, and provides guidance for the standing pose of the user 301 using:
“IsSamePlane (Vertical, head+feet+wrist, 20%) AND DistanceBetweenJoints (head, wrist1+wrist2 >20%) Guidance for standpose—none, Action—nextpose”.

If the user 301 is in the jump pose, the exercise counting repetition and form guidance system determines the relative positions of the user 301, determines the action to be performed, and provides form guidance to the user 301 using:
“If (IsRelativePosition (Above, head, wrist1+wrist2, 10%)) then Action—count increment”
Guidance for jumpPose “Ensure hands above head (isMinAngle (<30%, wrist1, head, wrist2) Feet wide apart (Is DistanceBetweenJoints (ankleL, ankleR, 20%))”

An example of an exercise language snippet executed by at least one processor of the exercise counting repetition and form guidance system describing the jumping jack exercise performed by the user 301 in real time is provided below:

```
“ex_id”: 1,
“ex_name”: “Jumping Jack”,
“number_of_poses”: 2,
“focus”: “cardio”,
“bodyarea”: [“core”, “lowerbody”],
“bodypart”: [“abs&core”, “fullbody”, “legs”, “butt”],
“secondary”: [“shoulder”, “abs&Core”, “back”, “legs”, “butt”],
“gender”: “both”,
“skills”: “intermediate”,
“weight”: False,
“siderepeat”: False,
“homeequipment”: False,
“rotation”: “front”,
“aimodel”: “model5”,
“count_json”: “jumping_jacks_count.json”,
“guidance_json”: “jumping_jacks_form_guidance.json”,
“cadence_json”: “jumping_jacks_cadence.json”,
“av_json”: “av/en/jumping_jacks/jumping_jacks_av_content.json”,
  “draw_joints”: [“head”, “wristl”, “wristr”, “anklel”, “ankler”],
“increment”: “count”,
“created_date”: “2018-07-31 : 11:07:01”,
  “modify_date”: “2018-07-31 : 11:07:01”
}
```

An example of an exercise language code snippet executed by at least one processor of the exercise counting repetition and form guidance system for counting the repetitions of the jumping jack exercise count performed by the user 301 in real time is provided below:

“function”: [

{

“id”: “1”,
“ex_id”: “1”,
“ex_name”: “Jumping Jack”,
“pose_id”: “1”,
“function”: “isrelativeposition”,
“position”: “above”,
“coordlist1”: “wristl,wristr”,
“coordlist2”: “head”,
“coordlist3”: null,
“calculation”: null,
“number”: “”,
“confidence_interval”: “10%”,
“angle”: “”,
“boolean”: “and”,
“result”: “count”,
“created_date”: “2018-12-16 : 12:12:59”,
“modify_date”: “2018-12-16 : 12:12:59”

},
{

“id”: “1”,
“ex_id”: “1”,
“ex_name”: “Jumping Jack”,
“pose_id”: “1”,
“function”: “isminangle”,
“position”: null,
“coordlist1”: “wristl”,
“coordlist2”: “head”,
“coordlist3”: “wristr”,
“calculation”: “lessthan”,
“number”: “”,
“confidence_interval”: “10%”,
“angle”: “30%”,
“boolean”: null,
“result”: “count”,
“created_date”: “2018-12-16 : 12:12:59”,
“modify_date”: “2018-12-16 : 12:12:59”

},
{

“id”: “2”,
“ex_id”: “1”,
“ex_name”: “Jumping Jack”,
“pose_id”: “2”,
“function”: “distancebetweenjoints”,
“position”: null,
“coordlist1”: “head”,
“coordlist2”: “wristl,wristr”,
“coordlist3”: null,
“calculation”: “greaterthan”,
“number”: “30%”,
“confidence_interval”: “10%”,
“angle”: “”,
“boolean”: “and”,
“result”: “nextpose”,
“created_date”: “2018-12-16 : 01:12:01”,
“modify_date”: “2018-12-16 : 01:12:01”

},
{

“id”: “3”,
“ex_id”: “1”,
“ex_name”: “Jumping Jack”,
“pose_id”: “2”,
“function”: “relativeposition”,
“position”: “above”,
“coordlist1”: “head”,
“coordlist2”: “wristl,wristr”,
“coordlist3”: null,
“calculation”: null,
“number”: “”,
“confidence_interval”: “10%”,
“angle”: “”,
“boolean”: null,
“result”: “nextpose”,
“created_date”: “2018-12-16 : 01:12:01”,
“modify_date”: “2018-12-16 : 01:12:01”

}

]

}

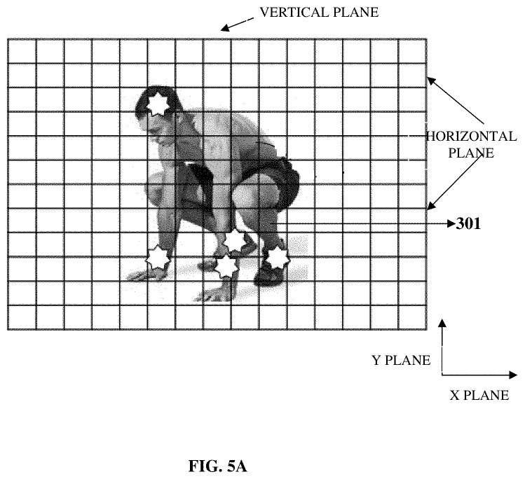

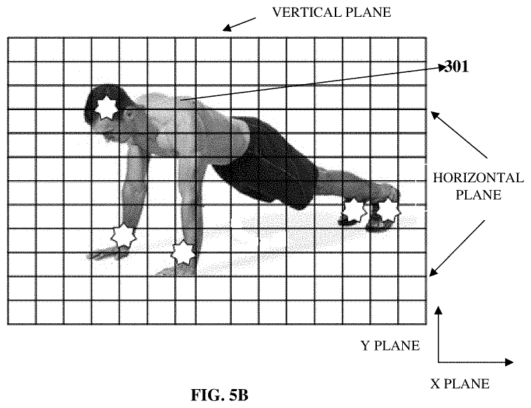

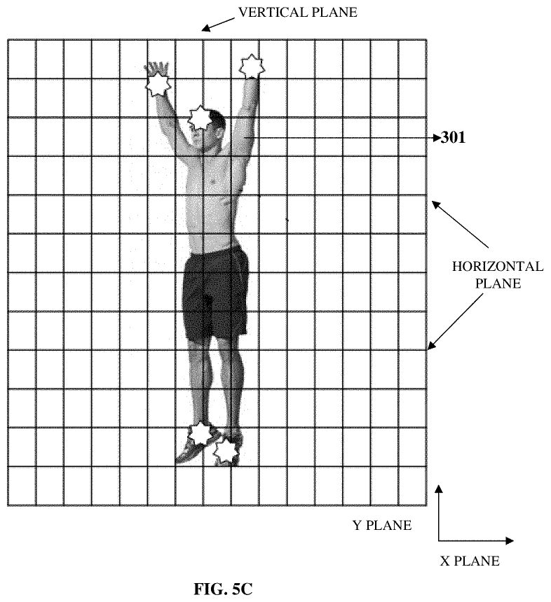

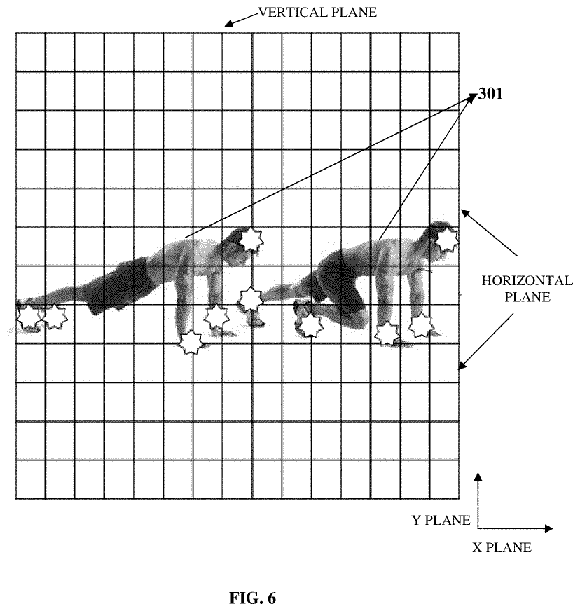

FIGS. 5A-5C exemplarily illustrates an embodiment of the exercise repetition and form guidance system where the user 301 performs a burpee exercise. The exercise counting repetition and form guidance system continuously and sequentially receives the captured consecutive exercising poses in real time from the capturing module of the electronic device. The exercise counting repetition and form guidance system continuously tracks the user 301 performing the burpee exercise and identifies the current three poses of the exercise performed by the user 301. The exercise counting repetition and form guidance system processes the received consecutive exercising poses of the user 301 using deep learning methods to derive a set of coordinates, for example, five or less on the X-Y coordinate planes as exemplarily illustrated in FIGS. 5A-5C.

FIG. 5A exemplarily illustrates the user 301 in a sitting pose for performing the specified exercise. FIG. 5B exemplarily illustrates the user 301 in a plank pose to perform the specified exercise. FIG. 5C exemplarily illustrates the user 301 in a jump pose to perform the specified exercise. The exercise counting repetition and form guidance system deployed on the electronic device normalizes the captured consecutive exercising poses of the user 301 based on the identified coordinates related to the consecutive exercising poses of the user 301. In an embodiment, the received consecutive exercising poses from the capturing module are, for example, the sitting pose of the user 301, the plank pose of the user 301, or the jump pose of the user 301 depending on the video frame at that particular instant. The normalization of the captured sitting pose of the user 301, plank pose of the user 301, or jump pose of the user 301 comprises mapping the height of the captured sitting pose of the user 301, plank pose of the user 301 or jump pose of the user 301 to a predefined standard height, for example, 6 feet and selectively mapping the captured sitting pose of the user 301, plank pose of the user 301 or jump pose of the user 301 to their corresponding rotational equivalents. The rotational equivalents correspond to the captured consecutive exercising poses of the user 301 viewed from an angle greater than 0 degrees and less or equal to 90 degrees, that is viewed from the side face of the user 301, because this angle provides the best view to enable counting the repetitions of the exercises performed by the user 301.

Following the process of normalization, the exercise counting repetition and form guidance system determines one or more relative positions of the user 301. For the specific burpee exercise, the exercise counting repetition and form guidance system determines the relative positions of the two wrists coordinates of the user 301 with respect to the head coordinates of the user 301. The determination of the relative positions of the two wrists coordinates of the user 301 with respect to the head coordinates of the user 301 comprises a confidence interval, for example, 10%. The following steps shows how each pose is detected in sequence and at the end of the last pose, for example, jump pose of the user 301, the count is incremented by the exercise counting repetition and form guidance system.

If the user 301 is in the sitting pose as exemplarily illustrated in FIG. 5A, the relative positions of the coordinates of the two wrists of the user 301 are identified below the head of the user 301 and the confidence interval of 10% is taken into account for further calculations by the exercise counting repetition and form guidance system. The plane of orientation of both the wrists of the user 301 and feet of the user 301 are in the same horizontal plane to further confirm that the user 301 has both hands and feet on the ground, considering 10% confidence interval. In addition, the distance between the wrists coordinates of the user 301 and feet coordinates of the user 301 are less than 50% of the height of the body of the user 301. The exercise counting repetition and form guidance system utilizes one or more instructions from the exercise language, for example, Instructionl AND Instruction 2 AND Instruction 3 to confirm whether the user 301 is in the sitting position with hands on the floor. Once the sitting position is detected, the exercise counting repetition and form guidance system then checks for the user 301 in the next pose of the sequence, that is, the plank pose of the user 301.

If the user 301 is in the plank pose as exemplarily illustrated in FIG. 5B, the relative positions of the coordinates of the two wrists of the user 301 are identified below the head of the user 301 and the confidence interval of 10% is taken into account for further calculations by the exercise counting repetition and form guidance system. The plane of orientation of both the wrists of the user 301 and feet of the user 301 are in the same horizontal plane to further confirm that the user 301 has both hands and feet on the ground considering 10% confidence interval. In addition, the distance between the wrists coordinates of the user 301 and feet coordinates of the user 301 are greater than 50% of the predefined height of the body of the user 301. The exercise counting repetition and form guidance system utilizes one or more instructions from the exercise language, for example, Instruction1 AND Instruction 2 AND Instruction 3 to confirm whether the user 301 is in the plank position. Once the plank position is detected, the exercise counting repetition and form guidance system then checks for the user 301 in the next pose of the sequence, that is, the jump pose of the user 301.

If the user 301 is in the jump pose as exemplarily illustrated in FIG. 5C, the relative positions of the coordinates of the two wrists of the user 301 are identified above the head of the user 301 and the confidence interval of 10% is taken into account for further calculations by the exercise counting repetition and form guidance system. The exercise counting repetition and form guidance system ensures appropriate form guidance if the hands of the user 301 touch each other. The form guidance check is provided using minimum angle instruction, that is the minimum angle between the wrists of the user 301 and neck of the user 301 is less than 20%, and the wrists of the user 301 are above the head of the user 301. The exercise counting repetition and form guidance system identifies that the jump pose of the user 301 is the final pose and allows the counter value to increase and provides form guidance to the user 301. In order to differentiate between the sitting pose of the user 301 and plank pose of the user 301, the exercise counting repetition and form guidance system further determines the relative positions of the feet coordinates of the user 301 with respect to the wrist coordinates of the user 301. If the user 301 performs the burpee exercise, the exercise counting repetition and form guidance system uses only three coordinates, for example, one for the head of the user 301, and two for the wrists of the user 301 to count the repetitions of the exercise by analyzing only two poses, namely, the plank pose of the user 301 and the jump pose of the user 301.

FIG. 6 exemplarily illustrates an embodiment of the exercise repetition and form guidance system where the user 301 performs a mountain climber exercise. The exercise counting repetition and form guidance system continuously receives the captured consecutive exercising poses in real time from the capturing module of the electronic device. The exercise counting repetition and form guidance system continuously tracks the user 301 performing the mountain climber exercise and identifies the current two poses of the exercise performed by the user 301. The exercise counting repetition and form guidance system process the received consecutive exercising poses of the user 301 using deep learning methods to derive a set of coordinates, for example, five or less on the X-Y coordinate planes as exemplarily illustrated in FIG. 6.

FIG. 6 exemplarily illustrates the user 301 in a plank pose and a mountain climber pose for performing the specified exercise in these poses. The exercise counting repetition and form guidance system deployed on the electronic device normalizes the captured consecutive exercising poses of the user 301 based on the identified coordinates related to the consecutive exercising poses of the user 301. In an embodiment, the received consecutive exercising poses from the capturing module are, for example, the plank pose of the user 301, and the mountain climber pose of the user 301 depending on the video frame at that particular instant. The normalization of the captured plank pose of the user 301, and the mountain climber pose of the user 301 comprise mapping the height of the captured plank pose of the user 301, and the mountain climber pose of the user 301 to a predefined standard height, for example, 6 feet and selectively mapping the captured plank pose of the user 301, and the mountain climber pose of the user 301 to their corresponding rotational equivalents. The rotational equivalents correspond to the captured plank pose of the user 301 and the mountain climber pose of the user 301 viewed from an angle greater than 0 degrees and less or equal to 90 degrees, that is viewed from the side face of the user 301, because this angle provides the best view to enable counting the repetitions of the exercises performed by the user 301.

Following the process of normalization, the exercise counting repetition and form guidance system calculates the relative distance between the coordinates of the feet of the user 301 and the head of the user 301. This step of calculating the relative distances between the coordinates of the feet of the user 301 and head of the user 301 comprise a confidence interval, for example, 15%. If the user 301 is in the plank position, the calculated relative distance between the coordinates of the feet of the user 301 and head of the user 301 is greater than 70% of the body height of the user 301 and are identified below 15% confidence interval, or another confidence interval is considered. The distance between the two feet of the user 301 is less than 5%. If the user 301 is in the mountain climber pose, the calculated relative distance between the coordinates of one of the feet of the user 301 and head of the user 301 are less than 70% and are identified with the above 15% confidence interval, or other confidence interval are considered to increase the counter value for the repetition of the exercise performed by the user 301.

If the user 301 is performing the mountain climber exercise, the exercise counting repetition and form guidance system uses only three coordinates, for example, one for head of the user 301, and two for feet of the user 301 to count the repetitions of the exercise by analyzing only the two poses, that is the plank pose of the user 301 and the mountain climber pose of the user 301. However, the feet of the user 301 and wrist coordinates of the user 301 are considered with or without the head coordinate to analyze the poses and count the repetitions of the exercise performed by the user 301.

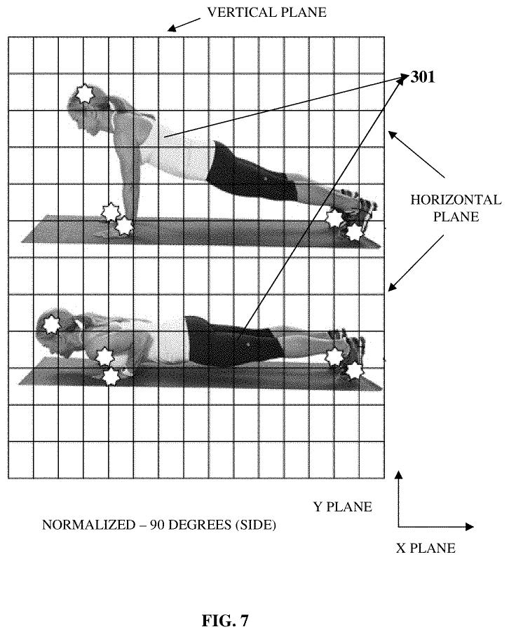

FIG. 7 exemplarily illustrates an embodiment of the exercise repetition and form guidance system where the user 301 performs exercises with pushups. The exercise counting repetition and form guidance system continuously receives the captured consecutive exercising poses in real time from the capturing module of the electronic device. The exercise counting repetition and form guidance system continuously tracks the user 301 performing the pushups exercise and identifies the current two poses of the exercise performed by the user 301. The exercise counting repetition and form guidance system processes the received consecutive exercising poses of the user 301 using deep learning methods to derive a set of coordinates, for example, five or less on the X-Y coordinate planes as exemplarily illustrated in FIG. 7.

FIG. 7 exemplarily illustrates the user 301 in a down pose and a plank pose to perform the specified exercise in the down pose and plank pose. The exercise counting repetition and form guidance system deployed on the electronic device normalizes the captured consecutive exercising poses of the user 301 based on the identified coordinates related to the consecutive exercising poses of the user 301. In an embodiment, the received consecutive exercising poses from the capturing module are, for example, the down pose of the user 301, and the plank pose of the user 301 depending on the video frame at that particular instant. The normalization of the captured down pose of the user 301, and the plank pose of the user 301 comprises mapping the height of the captured down pose of the user 301, and the plank pose of the user 301 to a predefined standard height, for example, 6 feet and selectively mapping the captured down pose of the user 301, and the plank pose of the user 301 to their corresponding rotational equivalents. The rotational equivalents correspond to the captured down pose of the user 301 and the plank pose of the user 301 viewed from an angle greater than 0 degrees and less or equal to 90 degrees, that is viewed from the side face of the user 301, because this angle provides the best view to enable counting the repetitions of the exercises performed by the user 301.

Following the process of normalization, the exercise counting repetition and form guidance system calculates the relative distance between the coordinates of one of the wrists of the user 301 and the head of the user 301. This step of calculating the relative distances between the coordinates of one of the wrists of the user 301 and head of the user 301 comprise a confidence interval, for example, 10%. If the user 301 is in the down position, the calculated relative distance between the coordinates of one of the wrists of the user 301 and head of the user 301 is less than 10% of the body height of the user 301. If the user 301 is in the plank pose, the calculated relative distance between the coordinates of one of the wrists of the user 301 and the head of the user 301 is greater than 20% of the body height of the user 301 and the relative positions of the head of the user 301 is above the wrists of the user 301. If the user 301 is performing the pushups exercise, the exercise counting repetition and form guidance system uses only three coordinates, for example, one for head of the user 301, and two for wrists of the user 301 to count the repetitions of the exercise by analyzing only the two poses, that is the down pose of the user 301 and the plank pose of the user 301.

The pseudo code below shows how the exercise language and the finite set of instructions/ functions can count the repetitions of the exercises and providing form guidance to the user 301 if the user 301 performs the pushups exercise. The exercise counting repetition and form guidance system normalizes the captured exercising poses of the user 301, for example, side pose of the user 301. The exercise counting repetition and form guidance system identifies thirteen of the received consecutive exercising poses of the user 301. If the user 301 is in the down pose, the exercise counting repetition and form guidance system determines the plane of orientation of the user 301, calculates the minimum angle between three of the identified coordinates of the user 301, and provides guidance for the down pose of the user 301 using:
“IsSamePlane (Horizontal, wrists+feet+head, 20%) AND isMinAngle (wrists, elbows, shoulders <40%) Guidance for DownPose Elbows behind wrist, wrists right under shoulders, and head above shoulders—(head should not sag)

If the user 301 is in the plank pose, the exercise counting repetition and form guidance system determines the plane of orientation of the user 301, determines the distance between the joints, and provides form guidance to the user301 using:
“IsSamePlane (Horizontal, wrists+feet, 10%) AND If (DistanceBetweenJoints (wrists1+wrists2, head) >15%) then count increment”
Guidance for PlankPose “Wrists, elbows right under shoulders, Head above shoulders—(head should not sag), No sagging hips, No raised hips”

If the user 301 is in the plank rotate pose, the exercise counting repetition and form guidance system determines the relative positions of the user 301, and provides form guidance to the user301 using:
“RelativePosition (Above, WristL, shoulders, 20%) AND
RelativePosition (Below, wristR, shoulders, 20%) OR
RelativePosition (Above, wristR, shoulders, 20%) AND
RelativePosition (Below, wristL, shoulders, 20%) then count increment”
Guidance “Keep wrist straight above the shoulders (IsSamePlane (Vertical, WristR, WristL, shoulders) AND IsRelativePosition (Above)
Keep wrist straight below the shoulder
No sagging hips (IsSamePlane (Angle, Shoulders, Waist, Ankles) AND
IsMinAngle (Shoulders, Waist, Ankles,170 degrees)”

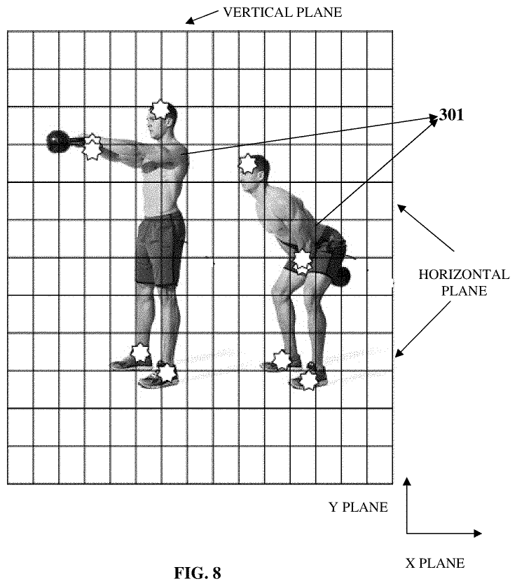

FIG. 8 exemplarily illustrates an embodiment of the exercise repetition and form guidance system where the user 301 performs an exercise of kettle bell swing. The exercise counting repetition and form guidance system continuously receives the captured consecutive exercising poses in real time from the capturing module of the electronic device. The exercise counting repetition and form guidance system continuously tracks the user 301 performing the kettle bell swing exercise and identifies the current two poses of the exercise performed by the user 301. The exercise counting repetition and form guidance system processes the received consecutive exercising poses of the user 301 using deep learning methods to derive a set of coordinates, for example, five or less on the X-Y coordinate planes as exemplarily illustrated in FIG. 8.

FIG. 8 exemplarily illustrates the user 301 in a standing pose and a swing pose to perform the specified exercise in the standing pose and the swing pose. The exercise counting repetition and form guidance system deployed on the electronic device normalizes the captured consecutive exercising poses of the user 301 based on the identified coordinates related to the consecutive exercising poses of the user 301. In an embodiment, the received consecutive exercising poses from the capturing module are, for example, the standing pose of the user 301, and the swing pose of the user 301 depending on the video frame at that particular instant. The normalization of the captured standing pose of the user 301, and the swing pose of the user 301 comprises mapping the height of the captured down pose of the user 301, and the swing pose of the user 301 to a predefined standard height, for example, 6 feet and selectively mapping the captured standing pose of the user 301, and the swing pose of the user 301 to their corresponding rotational equivalents. The rotational equivalents correspond to the captured standing pose of the user 301 and the swing pose of the user 301 viewed from an angle greater than 0 degrees and less or equal to 90 degrees, that is viewed from the side face of the user 301, because this angle provides the best view to enable counting the repetitions of the exercises performed by the user 301.

Following the process of normalization, the exercise counting repetition and form guidance system calculates the relative distance between the coordinates of the wrists of the user 301, for example, any one of the wrists of the user 301 as both the wrists of the user 301 comprises the same coordinates, and the head of the user 301. This step of calculating the relative distances between the coordinates of one of the wrists of the user 301 and head of the user 301 comprise a confidence interval, for example, 35%. If the user 301 is in the standing pose, the calculated relative distance between the coordinates of one of the wrists of the user 301 and head of the user 301 is greater than 35% of the body height of the user 301. If the user 301 is in the swing pose, the calculated relative distance between the coordinates of the two wrists of the user 301 are identified in front of the head of the user 301 upon considering the confidence interval for the counter value to increase to count the repetitions of the exercise performed by the user 301. If the user 301 is performing the kettle bell swing exercise, the exercise counting repetition and form guidance system uses only two to three coordinates, for example, one for head of the user 301 and one or two for wrists of the user 301 to count the repetitions of the exercise by analyzing the two poses, that is the standing pose of the user 301 and the swing pose of the user 301.

The pseudo code below shows how the exercise language and the finite set of instructions/ functions can count the repetitions of the exercises and providing form guidance to the user 301 if the user 301 performs the kettle bell swing exercise. The exercise counting repetition and form guidance system normalizes the captured exercising poses of the user 301, for example, side pose of the user 301.

If the user 301 is in the standing pose, the exercise counting repetition and form guidance system determines the distance between the body joints of the user 301, determines relative positions of the user 301, and provides guidance for the standing pose of the user 301 using:
“If (DistanceBetweenJoints (wristL+wristR, head) >35%) AND IsRelativePosition (above, head, wristL+wristR)”

If the user 301 is in the swing pose, the exercise counting repetition and form guidance system determines relative positions of the user 301, and provides guidance for the swing pose of the user 301 using:
“If (IsRelativePosition (Front, head, wrist+wrist2, 30%)) then count increment Guidance (are the hands straight or bent?)
IsMinAngle (shoulders, elbows, wrists, 170) AND
IsMaxAngle (shoulders, elbows, wrists, 170)”

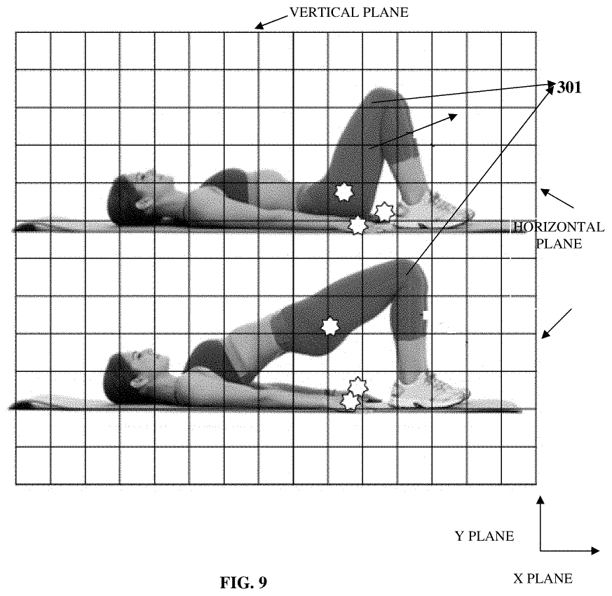

FIG. 9 exemplarily illustrates an embodiment of the exercise repetition and form guidance system where the user 301 performs a bridge exercise. The exercise counting repetition and form guidance system continuously receives the captured consecutive exercising poses in real time from the capturing module of the electronic device. The exercise counting repetition and form guidance system continuously tracks the user 301 performing the bridge exercise and identifies the current two poses of the exercise performed by the user 301. The exercise counting repetition and form guidance system processes the received consecutive exercising poses of the user 301 using deep learning methods to derive a set of coordinates, for example, five or less on the X-Y coordinate planes as exemplarily illustrated in FIG. 9.

FIG. 9 exemplarily illustrates the user 301 in a lie down pose and a lift up pose to perform the specified exercises for the lie down pose and the lift up pose. The exercise counting repetition and form guidance system deployed on the electronic device normalizes the captured consecutive exercising poses of the user 301 based on the identified coordinates related to the consecutive exercising poses of the user 301. In an embodiment, the received consecutive exercising poses from the capturing module are, for example, the lie down pose of the user 301, and the lift up pose of the user 301 depending on the video frame at that particular instant. The normalization of the captured lie down pose of the user 301, and the lift up pose of the user 301 comprises mapping the height of the captured lie down pose of the user 301, and the lift up pose of the user 301 to a predefined standard height, for example, 6 feet and selectively mapping the captured lie down pose of the user 301, and the lift up pose of the user 301 to their corresponding rotational equivalents. The rotational equivalents correspond to the captured lie down pose of the user 301 and the lift up pose of the user 301 viewed from an angle greater than 0 degrees and less or equal to 90 degrees, that is viewed from the side face of the user 301, because this angle provides the best view to enable counting the repetitions of the exercises performed by the user 301.

Following the process of normalization, the exercise counting repetition and form guidance system calculates the relative distance between the coordinates of the wrists of the user 301, for example, any one of the wrists of the user 301 as both the wrists of the user 301 comprises the same coordinates, and the hip of the user 301. This step of calculating the relative distances between the coordinates of one of the wrists of the user 301 and hip of the user 301 comprises a confidence interval, for example, 20%. If the user 301 is in the lie down pose, the calculated relative distance between the coordinates of one of the wrists of the user 301 and hip of the user 301 is less than 20% of the body height of the user 301. If the user 301 is in the lift up pose, the calculated relative distance between the coordinates of one of the wrists of the user 301 and hip of the user 301 are identified below 20% of the body height of the user 301 for the counter value to increase to count the repetitions of the exercise performed by the user 301.

If the user 301 is performing the bridge exercise, the exercise counting repetition and form guidance system uses only two to three coordinates, for example, one for hip of the user 301 and one or two for wrists of the user 301 to count the repetitions of the exercise by analyzing the two poses, that is the lie down pose of the user 301 and the lift up pose of the user 301. The bridge exercise uses only five or less coordinates irrespective of any specific body parts of the user 301. The bridge exercise makes use of coordinates pertaining to a group of coordinates from the wrists of the user 301 and hip of the user 301 unlike other exercises where the five or less coordinates belongs to a group of coordinates from the head of the user 301, wrists of the user 301 and feet of the user 301.

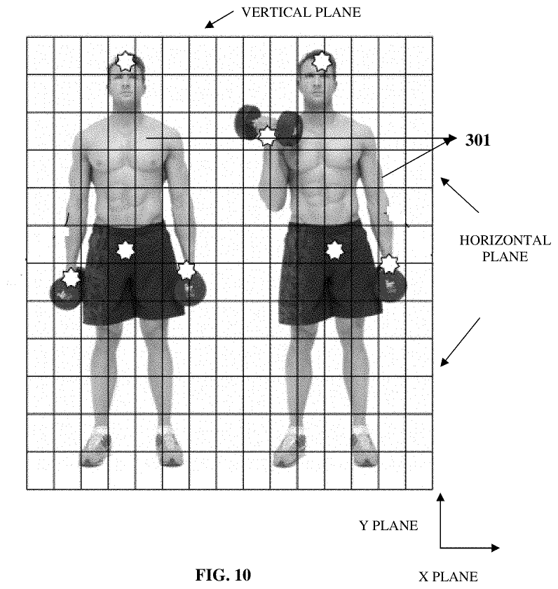

FIG. 10 exemplarily illustrates an embodiment of the exercise repetition and form guidance system where the user 301 performs weight lifting exercises. The exercise counting repetition and form guidance system continuously receives the captured consecutive exercising poses in real time from the capturing module of the electronic device. The exercise counting repetition and form guidance system continuously tracks the user 301 performing the weight lifting exercise and identifies the current two poses of the exercise performed by the user 301. The exercise counting repetition and form guidance system processes the received consecutive exercising poses of the user 301 using deep learning methods to derive a set of coordinates, for example, five or less that is specifically four coordinates on the X-Y coordinate planes as exemplarily illustrated in FIG. 10.

FIG. 10 exemplarily illustrates the user 301 in a standing pose and a weight lifting pose to perform the specified exercise. The exercise counting repetition and form guidance system deployed on the electronic device normalizes the captured consecutive exercising poses of the user 301 based on the identified coordinates related to the consecutive exercising poses of the user 301. In an embodiment, the received consecutive exercising poses from the capturing module are, for example, the standing pose of the user 301, and the weightlifting pose of the user 301 depending on the video frame at that particular instant. The normalization of the captured standing pose of the user 301, and the weightlifting pose of the user 301 comprises mapping the height of the captured standing pose of the user 301, and the weightlifting pose of the user 301 to a predefined standard height, for example, 6 feet and selectively mapping the captured standing pose of the user 301, and the weight lifting pose of the user 301 to their corresponding rotational equivalents. The rotational equivalents correspond to the captured standing pose of the user 301 and the weight lifting pose of the user 301 viewed from an angle of 0 degrees when viewed from the front face of the user 301, because this angle provides the best view to enable counting the repetitions of the exercises performed by the user 301.

Following the process of normalization, the exercise counting repetition and form guidance system calculates the relative positions of the two wrists coordinates of the user 301 with respect to hip coordinate of the user 301. This step of calculating the relative positions of the two wrists coordinates of the user 301 with respect to the hip coordinate of the user 301 comprises a confidence interval, for example, 10%. If the user 301 is in the standing pose, the relative positions of the coordinates of the two wrists of the user 301 are identified below the hip coordinate of the user 301 upon considering the confidence interval. Once the standing pose is detected, the exercise counting repetition and form guidance system then receives the next pose in the sequence, that is the weight lifting pose of the user 301. In the weight lifting pose, the relative positions of at least one coordinates of the two wrists of the user 301 are identified to be below the hip coordinate of the user 301 upon considering the 10% confidence interval for the counter value to increase to count the repetitions of the exercise performed by the user 301.

If the user 301 is performing the weight lifting exercise, the exercise counting repetition and form guidance system uses only three coordinates, for example, one for hip of the user 301 and two for wrists of the user 301 to count the repetitions of the exercise by analyzing the two poses, that is the standing pose of the user 301 and the weightlifting pose of the user 301. The weightlifting exercise uses only five or less coordinates irrespective of any specific body parts of the user 301. The weightlifting exercise makes use of coordinates pertaining to a group of coordinates from the wrists of the user 301 and hip of the user 301 unlike other exercises where the five or less coordinates belongs to a group of coordinates from head of the user 301, wrists of the user 301 and feet of the user 301. Further, in the weightlifting exercise, the exercise counting repetition and form guidance system determines relative positions of the coordinates of the two wrists of the user 301 and hip of the user 301 to increment the count, however one or other coordinates of the user, for example, head and wrist coordinates of the user 301 are used and the relative distance between the head coordinate of the user 301 and the wrist coordinates of the user 301 is calculated to further confirm the poses in sequence and increment the count.

The examples of the exercises illustrated in FIGS. 3-10 utilizes five or less coordinates to automatically count the repetitions of different forms of exercises and provides form guidance for a broad spectrum of exercises. The other examples of exercises for counting the repetitions of the exercises performed by the user comprises, squat, plank, crunch, scissors, side plank, boat, high knees, squat jump, side squat, etc. Also, the method disclosed herein employing the exercise counting repetition and form guidance system for counting repetitions of the exercises related to various other forms of kettlebell exercises, weights and yoga poses, for example clean, press, triceps, rows, pull, lunge, bicep curl, sun salutations, etc.

The pseudo code below shows how the exercise language and the finite set of instructions and functions can count the repetitions of the exercises and providing form guidance to the user 301 if the user 301 performs the abdomen crunch exercise. The exercise counting repetition and form guidance system normalizes the captured exercising poses of the user 301, for example, the side pose of the user 301. The exercise counting repetition and form guidance system identifies thirteen of the received consecutive exercising poses of the user 301.

If the user 301 is in the down pose, the exercise counting repetition and form guidance system determines the plane of orientation of the user, determines the relative positions of the user 301, and provides guidance for the down pose of the user 301 using:
“IsSamePlane (Horizontal, waist+feet+head+shoulders, 20%) AND
ifRelativePosition (above, knees, waist, >20%)
then nextpose
Guidance “wrists under head, Head not above shoulder by much”

If the user 301 is in the crunch pose, the exercise counting repetition and form guidance system determines the plane of orientation of the user, determines relative positions of the user 301, and provides guidance for the down pose of the user 301 using:
“IsSamePlane (Horizontal, waist+feet, 10%) AND

If (RelativePosition (Above, head, waist) >15%) then count increment Guidance “Head above but behind shoulders (that is do not pull your head using hands”

The pseudo code below shows how the exercise language and the finite set of instructions/ functions can count the repetitions of the exercises and provide form guidance to the user 301, if the user 301 performs the squat exercise. The exercise counting repetition and form guidance system normalizes the captured exercising poses of the user 301, for example, side pose of the user 301. The exercise counting repetition and form guidance system identifies thirteen of the received consecutive exercising poses of the user 301.

If the user 301 is in the squat pose, the exercise counting repetition and form guidance system determines the plane of orientation of the user, determines relative positions of the user 301, and provides guidance for the down pose of the user 301 using:
“IsSamePlane (Vertical, waist+shoulders+head, 10%) AND
RelativePosition (Front, knees, waist, 20%) AND
Relative Position (Above, waist, ankles, 10%)
Guidance “Knee behind ankle”

If the user 301 is in the standing pose, the exercise counting repetition and form guidance system determines the plane of orientation of the user, determines the distance between body joints of the user 301, and provides guidance for the down pose of the user 301 using:
“IsSamePlane (Vertical, waist+feet+knees+head, 20%) AND
DistanceBetweenJoints (Floor, feet, <10%) then count++
Guidance “stand straight”

The pseudo code below shows how the exercise language and the finite set of instructions/ functions can count the repetitions of the exercises and provide form guidance to the user 301 if the user 301 performs the plank exercise. The exercise counting repetition and form guidance system normalizes the captured exercising poses of the user 301, for example, side pose of the user 301. The exercise counting repetition and form guidance system identifies thirteen of the received consecutive exercising poses of the user 301.

If the user 301 is in the down pose, the exercise counting repetition and form guidance system determines the plane of orientation of the user, determines the relative positions of the user 301, and provides guidance for the down pose of the user 301 using:
“IsSamePlane (Vertical, wrists+head, 20%) AND
IsSamePlane (Horizontal, wrists+feet, 20%)
If (IsRelativePosition (Above, head, wrist1+wrist2, 30%)) count increment Guidance “Elbows right under shoulders, No sagging hips, No raised hips”

The pseudo code below shows how the exercise language and the finite set of instructions and functions can count the repetitions of the exercises and provide form guidance to the user 301 if the user 301 performs the lunge exercise. The exercise counting repetition and form guidance system normalizes the captured exercising poses of the user 301, for example, side pose of the user 301. The exercise counting repetition and form guidance system identifies thirteen of the received consecutive exercising poses of the user 301.

If the user 301 is in the lunge pose, the exercise counting repetition and form guidance system determines the plane of orientation of the user, determines the relative positions of the user 301, and provides guidance for the down pose of the user 301 using:
“IsSamePlane (Horizontal, waist+kneel, 10%) AND
RelativePosition (Front, kneel, waist, 20%) AND
RelativePosition (Front, waist, anklesR, 20%) OR
IsSamePlane (Horizontal, waist+KneeR, 10%) AND
RelativePosition (Front, kneeR, waist, 20%) AND
RelativePosition (Front, waist, anklesL, 20%)
Guidance “Knee should be behind ankle (RelativePosition (behind, kneel, ankleL))

If the user 301 is in the standing pose, the exercise counting repetition and form guidance system determines the plane of orientation of the user, determines the distance between body joints of the user 301, and provides guidance for the down pose of the user 301 using:

“IsSamePlane (Vertical, waist+feet+knees+head, 20%) AND
DistanceBetweenJoints (Floor, feet, <10%) then count increment
Guidance “stand straight”

The method disclosed herein utilizes minimal number of body coordinates to identify the consecutive exercising poses of the user and to determine the exercise count, and combines this information with an exercise language with a defined and finite set of algorithms and instructions to detect the exercising poses, provide form guidance and count the repetitions of the exercises performed by the user. The method using the exercise language enables diverse exercises to be developed without changing the code or the deep learning model, enables scalability and provides counting repetitions and form guidance for future exercises that are not available. The method enables the handheld user devices, for example, mobile phones with lower processing capability to count the repetitions of the exercise and provide form guidance with increased speed. Furthermore, the method enables processing of captured live video stream with fewer coordinates; hence the electronic devices processes higher image frames per second. Furthermore, the method does not send large amounts of video to a cloud environment or to the back-end data centers for analysis; hence the method saves on bandwidth. Furthermore, the method does not send video for analysis to cloud environment or data center, and waits for resulting analysis; hence the method is performed closer to real time. Furthermore, the optimization allows counting to be performed on mobile phones with standard cameras; the method allows the users who do not have access to expensive computing and powerful desktops or custom hardware with specialized cameras to use the method for counting the repetitions of the exercises. Furthermore, the method enables counting of the repetitions of exercises with estimation of time elapsed while performing the exercise; the data is used to generate informative reports and continual and near real time feedback, for example, information on amount of calorie burnt during the exercise, speed with which the user performed the exercises, encouragement to continue if the user is falling behind etc., and all the parameters helps the users to keep track of their physical health conditions.

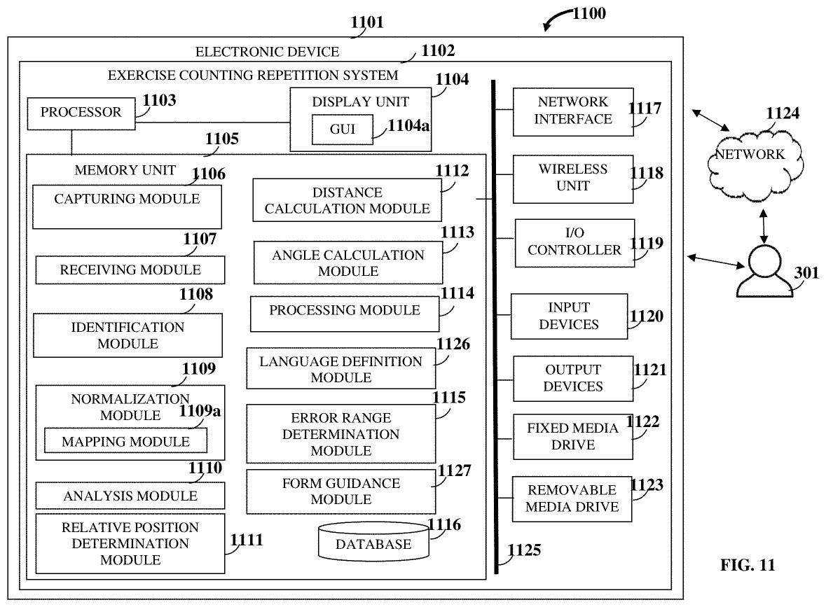

FIG. 11 exemplarily illustrates an architectural diagram of a system 1100 comprising an exercise counting repetition and form guidance system 1102 for automatically counting multiple repetitions and providing form guidance of an exercise performed by a user 301 in real time. The exercise counting repetition and form guidance system 1102 is a computer system that is programmable using a high-level computer programming language. In an embodiment, the exercise counting repetition and form guidance system 1102 uses programmed and purposeful hardware. The exercise counting repetition and form guidance system 1102 is implemented on a computing device, for example, a personal computer, a tablet computing device, a mobile computer, a portable computing device, a laptop, a touch device, a workstation, a server, portable electronic device, a network enabled computing device, an interactive network enabled communication device, any other suitable computing equipment, combinations of multiple pieces of computing equipment, etc. In an embodiment, the computing equipment is used to implement applications such as media playback applications, a web browser, an electronic mail (email) application, a calendar application, etc. In another embodiment, the computing equipment, for example, one or more servers are associated with one or more online services. In an embodiment, the exercise counting repetition and form guidance system 1102 is configured as a web-based platform, for example, a website hosted on a server or a network of servers.

The exercise counting repetition and form guidance system 1102 is installed on an electronic device 1101 via the network 1124, for example, a short-range network or a long-range network. The electronic device 1101 is, for example, personal computers, tablet computing devices, mobile computers, mobile phones, smartphones, portable computing devices, personal digital assistants, laptops, wearable computing devices such as the Google Glass® of Google Inc., the Apple Watch® of Apple Inc., etc., touch centric devices, client devices, portable electronic devices, network enabled computing devices, interactive network enabled communication devices, any other suitable computing equipment, combinations of multiple pieces of computing equipment, etc. In an embodiment, the electronic device 1101 is a hybrid computing device that combines the functionality of multiple devices. Examples of a hybrid computing device comprise a cellular telephone that includes media player functionality, a gaming device that includes a wireless communications capability, a cellular telephone that includes a document reader and multimedia functions, and a portable device that has network browsing, document rendering, and network communication capabilities. The electronic device 1101 further comprises multiple homogenous and/ or heterogeneous cores, multiple CPUs/processors of different kinds, special media, and other accelerators.

The network 1124 is, for example, the internet, an intranet, a wireless network, a communication network that implements Bluetooth® of Bluetooth Sig, Inc., a network that implements Wi-Fi® of Wi-Fi Alliance Corporation, an ultra-wideband communication network (UWB), a wireless universal serial bus (USB) communication network, a communication network that implements ZigBee® of ZigBee Alliance Corporation, a general packet radio service (GPRS) network, a mobile telecommunication network such as a global system for mobile (GSM) communications network, a code division multiple access (CDMA) network, a third generation (3G) mobile communication network, a fourth generation (4G) mobile communication network, a fifth generation (5G) mobile communication network, a long-term evolution (LTE) mobile communication network, a public telephone network, etc., a local area network, a wide area network, an internet connection network, an infrared communication network, etc., or a network formed from any combination of these networks. In an embodiment, the exercise counting repetition and form guidance system 1102 is accessible to the satellite internet of users, for example, through a broad spectrum of technologies and devices such as cellular phones, tablet computing devices, etc., with access to the internet.

As exemplarily illustrated in FIG. 11, the exercise counting repetition and form guidance system 1102 comprises a non-transitory computer readable storage medium, for example, a memory unit 1105 for storing programs and data, and at least one processor 1103 communicatively coupled to the non-transitory computer readable storage medium. As used herein, “non-transitory computer readable storage medium” refers to all computer readable media, for example, non-volatile media, volatile media, and transmission media, except for a transitory, propagating signal. Non-volatile media comprise, for example, solid state drives, optical discs or magnetic disks, and other persistent memory volatile media including a dynamic random access memory (DRAM), which typically constitute a main memory. Volatile media comprise, for example, a register memory, a processor cache, a random access memory (RAM), etc. Transmission media comprise, for example, coaxial cables, copper wire, fiber optic cables, modems, etc., including wires that constitute a system bus coupled to the processor 1103. The non-transitory computer readable storage medium is configured to store computer program instructions defined by modules, for example, 1106, 1107, 1108, 1109, 1109a, 1110, 1111, 1112, 1113, 1114, 1115, 1126, 1127 etc., of the exercise counting repetition and form guidance system 1102. The modules 1106, 1107, 1108, 1109, 1109a, 1110, 1111, 1112, 1113, 1114, 1115 1126, and 1127 are installed and stored in the memory unit 1105 of the exercise counting repetition and form guidance system 1102. The memory unit 1105 is used for storing program instructions, applications, and data. The memory unit 1105 is, for example, a random access memory (RAM) or another type of dynamic storage device that stores information and instructions for execution by the processor 1103. The memory unit 1105 also stores temporary variables and other intermediate information used during execution of the instructions by the processor 1103. The exercise counting repetition and form guidance system 1102 further comprises a read only memory (ROM) or another type of static storage device that stores static information and instructions for the processor 1103.

The processor 1103 is configured to execute the computer program instructions defined by the modules, for example, 1106, 1107, 1108, 1109, 1109a, 1110, 1111, 1112, 1113, 1114, 1115, 1126, 1127 etc., of the exercise counting repetition and form guidance system 1102. The processor 1103 refers to any of one or more microprocessors, central processing unit (CPU) devices, graphical processing units (GPU) devices, finite state machines, computers, microcontrollers, digital signal processors, logic, a logic device, an user circuit, an application specific integrated circuit (ASIC), a field-programmable gate array (FPGA), a chip, etc., or any combination thereof, capable of executing computer programs or a series of commands, instructions, or state transitions. In an embodiment, the processor 1103 is implemented as a processor set comprising, for example, a programmed microprocessor and a math or graphics co-processor. The processor 1103 is selected, for example, RISC based computer processors of ARM Holdings, Motorola® processors, Qualcomm® processors, etc. The exercise counting repetition and form guidance system 1102 disclosed herein is not limited to employing a processor 1103. In an embodiment, the exercise counting repetition and form guidance system 1102 employs a controller or a microcontroller. Any logical and arithmetic operations involved in the execution of the instructions are computed using arithmetic and logic unit (not shown) and graphical processor unit (not shown). In another embodiment, specialized hardware for processing the video images and specialized systems for machine learning, various networking devices or external input/output (I/0) devices are also employed to support the implementation through the networking unit and external input/output (I/0) devices.

As exemplarily illustrated in FIG. 11, the exercise counting repetition and form guidance system 1102 further comprises a data bus 1125, a network interface 1117, an input/output (I/0) controller 1119, input devices 1120, a fixed media drive 1122 such as a hard drive, a removable media drive 1123 for receiving removable media, output devices 1121, etc. The data bus 1125 permits communications between the modules, for example, 1106, 1107, 1108, 1109, 1109a, 1110, 1111, 1112, 1113, 1114, 1115, 1126, 1127 etc., of the exercise counting repetition and form guidance system 1102. The network interface 1117 enables connection of the exercise counting repetition and form guidance system 1102 to the network 1124. In an embodiment, the network interface 1117 is provided as an interface card also referred to as a line card. The network interface 1117 comprises, for example, one or more of an infrared (IR) interface, an interface implementing Wi-Fi® of Wi-Fi Alliance Corporation, a universal serial bus (USB) interface, a FireWire® interface of Apple Inc., an Ethernet interface, a frame relay interface, a cable interface, a digital subscriber line (DSL) interface, a token ring interface, a peripheral controller interconnect (PCI) interface, a local area network (LAN) interface, a wide area network (WAN) interface, interfaces using serial protocols, interfaces using parallel protocols, Ethernet communication interfaces, asynchronous transfer mode (ATM) interfaces, a high speed serial interface (HSSI), a fiber distributed data interface (FDDI), interfaces based on a transmission control protocol (TCP)/internet protocol (IP), interfaces based on wireless communications technology such as satellite technology, radio frequency (RF) technology, near field communication, etc. The I/0 controller 1119 controls input actions and output actions performed by the exercise counting repetition and form guidance system 1102.

The display screen 1104, via the graphical user interface (GUI) 1104a, displays the number of repetitions of the exercises performed or calorie burnt, time elapsed during an exercise, speed of performing exercises, halt times, etc. The display screen 1104 is, for example, a video display, a liquid crystal display, a plasma display, an organic light emitting diode (OLED) based display, etc. The exercise counting repetition and form guidance system 1102 provides the GUI 1104a on the display screen 1104. The GUI 1104a is, for example, an online web interface, a web based downloadable application interface, a mobile based downloadable application interface, etc. The display screen 1104 displays the GUI 1104a. The input devices 1120 are used for inputting data into the exercise counting repetition and form guidance system 1102. The input commands, are for example, selection or navigation through user interfaces, selection of specific exercise that the user intends to perform, etc. The input devices 1120 are, for example, a keyboard such as an alphanumeric keyboard, a microphone, a joystick, a pointing device such as a computer mouse, a touch pad, a light pen, a physical button, a touch sensitive display device, a track ball, a pointing stick, any device capable of sensing a tactile input, etc.

The output devices 1121 output the results of operations performed by the exercise counting repetition and form guidance system 1102.

The modules of the exercise counting repetition and form guidance system 1102 comprise a capturing module 1106, a receiving module 1107, a language definition module 1126, an identification module 1108, a normalization module 1109, an analysis module 1110, a relative position determination module 1111, a distance calculation module 1112, an angle calculation module 1113, a processing module 1114, an error range determination module 1105, and a form guidance module 1127 stored in the memory unit 1105 of the exercise counting repetition and form guidance system 1102. The capturing module 1106 captures one or more consecutive exercising poses of the exercise, performed by the user 301, in real time as a video stream. The capturing module 1106 eliminates the requirement for high bandwidth for streaming the captured consecutive exercising poses of the user 301 for analysis aided by large computing environments. The receiving module 1107 receives the captured consecutive exercising poses in real time, from the capturing module 1106. The language definition module 1126 defines an exercise language comprising a finite set of instructions for automatically counting the repetitions of the exercise and providing form guidance to the user based on the received consecutive exercising poses. The language definition module automatically counts the repetitions and provides form guidance for any type of exercise without changing the deep learning methods and the codes used in the exercise counting repetition and form guidance system 1102.

The identification module 1108 identifies multiple coordinates corresponding to multiple specific body joints of the user 301 related to the consecutive exercising poses using the defined exercise language based on the received consecutive exercising poses of the user 301. The identification module 1108 for identifying the coordinates of the consecutive exercising poses of the user 301 comprises a convolutional neural network (CNN) and deep learning algorithms. The identification module 1108 uses the convolutional neural network (CNN) and the deep learning algorithms for detecting image and video objects, human pose estimation, and computer vision analysis.

The normalization module 1109 normalizes the captured consecutive exercising poses of the user 301 based on the identified coordinates related to the consecutive exercising poses. The analysis module 1110 analyzes the identified coordinates associated with the normalized consecutive exercising poses of the user 301 for determining a plane of orientation of the user 301. The normalization module 1109 further comprises a mapping module 1109a for mapping the height of the captured consecutive exercising poses of the user 301 to a predefined standard height of the user 301, and selectively mapping the captured consecutive exercising poses of the user 301 to their corresponding rotational equivalents.

The relative position determination module 1111 determines one or more relative positions of the user 301 based on the determination of the plane of orientation of the consecutive exercising poses of the user 301. The distance calculation module 1112 calculates a distance between at least two of the identified coordinates of the consecutive exercising poses of the user 301 based on the determined plane of orientation and the determined relative positions of the identified coordinates of the consecutive exercising poses of the user 301 as a percentage of the predefined height. The angle calculation module 1113 calculates a maximum angle and a minimum angle between three of the identified coordinates of the consecutive exercising poses of the user 301 based on the calculated distance between at least two of the identified coordinates of the consecutive exercising poses of the user 301. The processing module 1114 processes the received consecutive exercising poses of the user 301 by utilizing one or more stored instructions in the exercise counting repetition and form guidance system 1102 from the defined exercise language based on the calculated maximum angle and the minimum angle between three of the identified coordinates of the consecutive exercising poses of the user 301 thereby automatically counting the repetitions of an exercise performed by the user 301. The analysis module 1110, the relative position determination module 1111, the distance calculation module 1112, and the angle calculation module 1113 further comprise an error range determination module 1115 for determining an acceptable error range of a predefined confidence interval. The analysis module 1110 determines the plane of orientation of the consecutive exercising poses for the predefined exercises performed in sequence by the user 301 corresponds to specific detected points of the user 301. The form guidance module 1127 provides appropriate exercise form guidance to the user for performing the exercise in real time based on the processed consecutive exercising poses of the user by verifying the exercise being performed by the user is in the appropriate form.

The exercise counting repetition and form guidance system 1102 stores the exercise language instructions in a database 1116 of the exercise counting repetition and form guidance system 1102. The database 1116 of the exercise counting repetition and form guidance system 1102 can be any storage area or medium that can be used for storing data and files. In an embodiment, the exercise counting repetition and form guidance system 1102 stores the received information in external databases, for example, a structured query language (SQL) data store or a not only SQL (NoSQL) data store such as the Microsoft® SQL Server®, the Oracle® servers, the MySQL® database of MySQL AB Company, the mongoDB® of MongoDB, Inc., the Neo4j graph database of Neo Technology Corporation, the Cassandra database of the Apache Software Foundation, the HBase™ database of the Apache Software Foundation, etc. In another embodiment, the database 1116 can be a location on a file system. In another embodiment, the database 1116 can be remotely accessed by the exercise counting repetition and form guidance system 1102 via the network 1124. In another embodiment, the database 1116 is configured as a cloud based database implemented in a cloud computing environment, where computing resources are delivered as a service over the network 1124.

Computer applications and programs are used for operating the modules of the exercise counting repetition and form guidance system 1102. The programs are loaded onto the fixed media drive 1122 and into the memory unit 1105 of the exercise counting repetition and form guidance system 1102 via the removable media drive 1123. In an embodiment, the computer applications and programs are loaded directly on the exercise counting repetition and form guidance system 1102 via the network 1124. The processor 1103 executes an operating system, for example, the Linux® operating system, the Unix® operating system, any version of the Microsoft® Windows® operating system, the Mac OS of Apple Inc., the IBM® OS/2, VxWorks® of Wind River Systems, Inc., QNX Neutrino® developed by QNX Software Systems Ltd., the Palm OS®, the Solaris operating system developed by Sun Microsystems, Inc., etc. The exercise counting repetition and form guidance system 1102 employs the operating system for performing multiple tasks. The operating system is responsible for management and coordination of activities and sharing of resources of the exercise counting repetition and form guidance system 1102. The operating system further manages security of the exercise counting repetition and form guidance system 1102, peripheral devices connected to the exercise counting repetition and form guidance system 1102, and network connections. The operating system employed on the exercise counting repetition and form guidance system 1102 recognizes, for example, inputs provided by a user of the exercise counting repetition and form guidance system 1102 using one of the input devices 1120, the output devices 1121, files, and directories stored locally on the fixed media drive 1122. The operating system on the exercise counting repetition and form guidance system 1102 executes different programs using the processor 1103. The processor 1103 and the operating system together define a computer platform for which application programs in high level programming languages are written.

The processor 1103 of the exercise counting repetition and form guidance system 1102 retrieves instructions defined by the capturing module 1106, the receiving module 1107, the language definition module 1126, the identification module 1108, the normalization module 1109, the analysis module 1110, the relative position determination module 1111, the distance calculation module 1112, the angle calculation module 1113, the processing module 1114, and the error range determination module 1115 for performing respective functions disclosed above. The processor 1103 retrieves instructions for executing the modules, for example, 1106, 1107, 1108, 1109, 1110, 1111, 1112, 1113, 1114, 1115, 1126, 1127, etc., of the exercise counting repetition and form guidance system 1102 from the memory unit 1105. A program counter determines the location of the instructions in the memory unit 1105. The program counter stores a number that identifies the current position in the program of each of the modules, for example, 1106, 1107, 1108, 1109, 1110, 1111, 1112, 1113, 1114, 1115, 1126, 1127,etc., of the exercise counting repetition and form guidance system 1102. The instructions fetched by the processor 1103 from the memory unit 1105 after being processed are decoded. The instructions are stored in an instruction register in the processor 1103. After processing and decoding, the processor 1103 executes the instructions, thereby performing one or more processes defined by those instructions.

At the time of execution, the instructions stored in the instruction register are examined to determine the operations to be performed. The processor 1103 then performs the specified operations. The operations comprise arithmetic operations and logic operations. The operating system performs multiple routines for performing a number of tasks required to assign the input devices 1120, the output devices 1121, and the memory unit 1105 for execution of the modules, for example, 1106, 1107, 1108, 1109, 1110, 1111, 1112, 1113, 1114, 1115, 1126, 1127 etc., of the exercise counting repetition and form guidance system 1102. The tasks performed by the operating system comprise, for example, assigning memory to the modules, for example, 1106, 1107, 1108, 1109, 1110, 1111, 1112, 1113, 1114, 1115, 1126 1127, etc., of the exercise counting repetition and form guidance system 1102 and to data used by the exercise counting repetition and form guidance system 1102, moving data between the memory unit 1105 and disk units, and handling input/output operations. The operating system performs the tasks on request by the operations and after performing the tasks, the operating system transfers the execution control back to the processor 1103. The processor 1103 continues the execution to obtain one or more outputs. The outputs of the execution of the modules, for example, 1106, 1107, 1108, 1109, 1110, 1111, 1112, 1113, 1114, 1115, 1126, 1127, etc., of the exercise counting repetition and form guidance system 1102 are displayed to a user of the exercise counting repetition and form guidance system 1102 on the output device 1121. In an embodiment, one or more portions of the exercise counting repetition and form guidance system 1102 are distributed across one or more computer systems (not shown) coupled to the network 1124.

The non-transitory computer readable storage medium having embodied thereon, computer program codes comprising instructions executable by at least one processor 1103 for automatically counting multiple repetitions and providing form guidance of an exercise performed by the user in real time. The computer program codes comprise a first computer program code for capturing one or more consecutive exercising poses of the exercise performed by the user 301 , in real time as a video stream by a capturing module of an electronic device 1101; a second computer program code for receiving the captured consecutive exercising poses in real time, from the capturing module 1106 of the electronic device 1101; a third computer program code for defining an exercise language comprising a finite set of instructions for automatically counting the repetitions of the exercise and providing form guidance to the user based on the received consecutive exercising poses; a fourth computer program code for identifying multiple coordinates corresponding to multiple specific body joints of the user 301 related to the consecutive exercising poses using the defined exercise language based on the received consecutive exercising poses of the user 301; a fifth computer program code for normalizing the captured consecutive exercising poses of the user 301 based on the identified coordinates related to the consecutive exercising poses; a sixth computer program code for analyzing the identified coordinates associated with the normalized consecutive exercising poses of the user 301 for determining a plane of orientation of the user 301; a seventh computer program code for determining one or more relative positions of the user 301 based on the determination of the plane of orientation of the consecutive exercising poses of the user 301, wherein the relative positions corresponds to a position between and among the identified coordinates of the consecutive exercising poses of the user 301; an eighth computer program code for calculating a distance between at least two of the identified coordinates of the consecutive exercising poses of the user 301 based on the determined plane of orientation and the determined relative positions of the identified coordinates of the consecutive exercising poses of the user 301 as a percentage of the predefined height; an ninth computer program code for calculating a maximum angle and a minimum angle between three of the identified coordinates of the consecutive exercising poses of the user 301 based on the calculated distance between at least two of the identified coordinates of the consecutive exercising poses of the user 301; a tenth computer program code for processing the received consecutive exercising poses of the user by utilizing one or more stored instructions in the exercise counting repetition and form guidance system 1102 from the defined exercise language based on the calculated maximum angle and the minimum angle between three of the identified coordinates of the consecutive exercising poses of the user 301 thereby automatically counting the repetitions of an exercise performed by the user 301, and a eleventh computer program code for providing an appropriate exercise form guidance to the user performing the exercise in real time based on the processed consecutive exercising poses of the user by verifying the exercise being performed by the user is in the appropriate form.

The non-transitory computer readable storage medium, wherein the fifth computer program code further comprises a twelfth computer program code for mapping a height of the captured consecutive exercising poses of the user 301 to a predefined standard height of the user 301, and selectively mapping the captured consecutive exercising poses of the user 301 to their corresponding rotational equivalents. The non-transitory computer readable storage medium further comprises a thirteenth computer program code for determining one or more calorie counts of the user 301, the efforts and intensity of the user 301 during exercise routines, the speed of performing the exercise by the user 301, and the halt times based on the automatic counting of the repetitions of exercises and the time elapsed performing an exercise, when the exercise is being done by the user 301 and providing instant feedback and appropriate exercise form guidance to the user 301. The non-transitory computer readable storage medium further comprises a fourteenth computer program code for determining an acceptable error range for a predefined confidence interval of the exercise performed by the user 301.

It will be readily apparent in different embodiments that the various methods, algorithms, and computer programs disclosed herein are implemented on non-transitory computer readable storage media appropriately programmed for computing devices. The non-transitory computer readable storage media participates in providing data, for example, instructions that are read by a computer, a processor or a similar device. In different embodiments, the “non-transitory computer readable storage media” further refers to a single medium or multiple media, for example, a centralized database, a distributed database, and/or associated caches and servers that store one or more sets of instructions that are read by a computer, a processor or a similar device. The “non-transitory computer readable storage media” further refers to any medium capable of storing or encoding a set of instructions for execution by a computer, a processor or a similar device and that causes a computer, a processor or a similar device to perform any one or more of the methods disclosed herein. Common forms of non-transitory computer readable storage media comprise, for example, a floppy disk, a flexible disk, a hard disk, magnetic tape, a laser disc, a Blu-ray Disc® of the Blu-ray Disc Association, any magnetic medium, a compact disc-read only memory (CD-ROM), a digital versatile disc (DVD), any optical medium, a flash memory card, punch cards, paper tape, any other physical medium with patterns of holes, a random access memory (RAM), a programmable read only memory (PROM), an erasable programmable read only memory (EPROM), an electrically erasable programmable read only memory (EEPROM), a flash memory, any other memory chip or cartridge, or any other medium from which a computer can read.

In an embodiment, the computer programs that implement the methods and algorithms disclosed herein are stored and transmitted using a variety of media, for example, the computer readable media in a number of manners. In an embodiment, hard-wired circuitry or custom hardware is used in place of, or in combination with, software instructions for implementing the processes of various embodiments. Therefore, the embodiments are not limited to any specific combination of hardware and software. The computer program codes comprising computer executable instructions can be implemented in any programming language. Examples of programming languages that can be used comprise C, C++, C#, Java®, JavaScript®, Fortran, Ruby, Perl®, Python®, Visual Basic®, hypertext preprocessor (PHP), Microsoft® .NET, Objective-C®, etc. Other object-oriented, functional, scripting, and/or logical programming languages can also be used. In an embodiment, the computer program codes or software programs are stored on or in one or more mediums as object code. In another embodiment, various aspects of the computer implemented method and the exercise counting repetition and form guidance system 1102 disclosed herein are implemented in a non-programmed environment comprising documents created, for example, in a hypertext markup language (HTML), an extensible markup language (XML, JSON), or other format that render aspects of a graphical user interface (GUI) or perform other functions, when viewed in a visual area or a window of a browser program. In another embodiment, various aspects of the computer implemented method and the exercise counting repetition and form guidance system 1102 disclosed herein are implemented as programmed elements, or non-programmed elements, or any suitable combination thereof.

Where databases are described such as the database 1116, it will be understood by one of ordinary skill in the art that (i) alternative database structures to those described may be employed, and (ii) other memory structures besides databases may be employed. Any illustrations or descriptions of any sample databases disclosed herein are illustrative arrangements for stored representations of information. In an embodiment, any number of other arrangements are employed besides those suggested by tables illustrated in the drawings or elsewhere. Similarly, any illustrated entries of the databases represent exemplary information only; one of ordinary skill in the art will understand that the number and content of the entries can be different from those disclosed herein. In another embodiment, despite any depiction of the databases as tables, other formats including relational databases, object-based models, and/or distributed databases are used to store and manipulate the data types disclosed herein. Object methods or behaviors of a database can be used to implement various processes such as those disclosed herein. In another embodiment, the databases are, in a known manner, stored locally or remotely from a device that accesses data in such a database. In embodiments where there are multiple databases in the exercise counting repetition and form guidance system 1102, the databases are integrated to communicate with each other for enabling simultaneous updates of data linked across the databases, when there are any updates to the data in one of the databases.

The computer implemented method and the exercise counting repetition and form guidance system 1102 disclosed herein can be configured to work in a network environment comprising one or more computers that are in communication with one or more devices via a network. In an embodiment, the computers communicate with the devices directly or indirectly, via a wired medium or a wireless medium such as the Internet, a local area network (LAN), a wide area network (WAN) or the Ethernet, a token ring, or via any appropriate communications mediums or combination of communications mediums. Each of the devices comprises processors, examples of which are disclosed above, that are adapted to communicate with the computers. In an embodiment, each of the computers is equipped with a network communication device, for example, a network interface card, a modem, or other network connection device suitable for connecting to a network. Each of the computers and the devices executes an operating system, examples of which are disclosed above. While the operating system may differ depending on the type of computer, the operating system provides the appropriate communications protocols to establish communication links with the network. Any number and type of machines may be in communication with the computers.

The computer implemented method and the exercise counting repetition and form guidance system 1102 disclosed herein are not limited to a particular computer system platform, processor, operating system, or network. In an embodiment, one or more aspects of the computer implemented method and the exercise counting repetition and form guidance system 1102 disclosed herein are distributed among one or more computer systems, for example, servers configured to provide one or more services to one or more client computers, or to perform a complete task in a distributed system. For example, one or more aspects of the computer implemented method and the exercise counting repetition and form guidance system 1102 disclosed herein are performed on a client-server system that comprises components distributed among one or more server systems that perform multiple functions according to various embodiments. These components comprise, for example, executable, intermediate, or interpreted code, which communicate over a network using a communication protocol. The computer implemented method and the exercise counting repetition and form guidance system 1102 disclosed herein are not limited to be executable on any particular system or group of systems, and are not limited to any particular distributed architecture, network, or communication protocol.

The foregoing examples have been provided merely for explanation and are in no way to be construed as limiting of the method and the exercise counting repetition and form guidance system 1102 disclosed herein. While the method and the exercise counting repetition and form guidance system 1102 have been described with reference to various embodiments, it is understood that the words, which have been used herein, are words of description and illustration, rather than words of limitation. Furthermore, although the method and the exercise counting repetition and form guidance system 1102 have been described herein with reference to particular means, materials, and embodiments, the method and the exercise counting repetition and form guidance system 1102 are not intended to be limited to the particulars disclosed herein; rather, the method and the exercise counting repetition and form guidance system 1102 extend to all functionally equivalent structures, methods and uses, such as are within the scope of the appended claims. While multiple embodiments are disclosed, it will be understood by those skilled in the art, having the benefit of the teachings of this specification, that the method and the exercise counting repetition and form guidance system 1102 disclosed herein are capable of modifications and other embodiments may be effected and changes may be made thereto, without departing from the scope and spirit of the method and the exercise counting repetition and form guidance system 1102 disclosed herein.

We claim:

1. A method for automatically counting a plurality of repetitions and providing form guidance for an exercise performed by a user in real time, the method employing an exercise counting repetition and form guidance system comprising at least one processor configured to execute computer program instructions for performing the method comprising:
capturing one or more consecutive exercising poses of the exercise, performed by the user, in real time as a video stream by a capturing module of an electronic device by the exercise counting repetition and form guidance system;
receiving the captured consecutive exercising poses in real time, from the capturing module of the electronic device, by the exercise counting repetition and form guidance system;
defining an exercise language comprising a finite set of instructions for automatically counting the repetitions of the exercise and providing form guidance to the user based on the received consecutive exercising poses by the exercise counting repetition and form guidance system;
identifying a plurality of coordinates corresponding to a plurality of specific body joints of the user related to the consecutive exercising poses using the defined exercise language based on the received consecutive exercising poses of the user by the exercise counting repetition and form guidance system;
normalizing the captured consecutive exercising poses of the user based on the identified coordinates related to the consecutive exercising poses by the exercise counting repetition and form guidance system;
analyzing the identified coordinates associated with the normalized consecutive exercising poses of the user for determining a plane of orientation of the user by the exercise counting repetition and form guidance system;
determining one or more relative positions of the user based on the determination of the plane of orientation of the consecutive exercising poses of the user, wherein the relative positions corresponds to a position between and among the identified coordinates of the consecutive exercising poses of the user by the exercise counting repetition and form guidance system;
calculating a distance between at least two of the identified coordinates of the consecutive exercising poses of the user based on the determined plane of orientation and the determined one or more relative positions of the identified coordinates of the consecutive exercising poses of the user as a percentage of the predefined height by the exercise counting repetition and form guidance system;
calculating a maximum angle and a minimum angle between three of the identified coordinates of the consecutive exercising poses of the user based on the calculated distance between at least two of the identified coordinates of the consecutive exercising poses of the user by the exercise counting repetition and form guidance system;
processing the received consecutive exercising poses of the user by utilizing one or more stored instructions in the exercise counting repetition and form guidance system from the defined exercise language based on the calculated maximum angle and the minimum angle between three of the identified coordinates of the consecutive exercising poses of the user thereby automatically counting the repetitions and providing form guidance of an exercise performed by the user by the exercise counting repetition and form guidance system; and
providing an appropriate exercise form guidance to the user performing the exercise in real time based on the processed consecutive exercising poses of the user by verifying the exercise being performed by the user is in the appropriate form by the exercise counting repetition and form guidance system.

2. The method of claim 1, wherein identifying the coordinates of the consecutive exercising poses of the user comprises using a Convolutional Neural Network (CNN) and deep learning algorithms for detecting image and video objects, human pose estimation, and computer vision analysis.

3. The method of claim 1, where in the defined exercise language with a finite set of instructions in the exercise counting repetition and form guidance system automatically counts the repetitions and provides form guidance for a plurality of exercises using the deep learning methods and the finite set of instructions used in the exercise counting repetition and form guidance system.

4. The method of claim 1, wherein the normalization of the consecutive exercising poses of the user comprises mapping a height of the captured consecutive exercising poses of the user to a predefined standard height of the user, and selectively mapping the captured consecutive exercising poses of the user to their corresponding rotational equivalents.

5. The method of claim 1, wherein the on-device analysis of captured consecutive exercising poses of the user eliminates the requirement for high bandwidth for streaming the captured consecutive exercising poses of the user for analysis aided by large computing environments.

6. The method of claim 1, further processes higher image frames per second of the captured consecutive exercising poses of the user thereby performing fast moving exercises.

7. The method of claim 1, further comprises determining one or more calorie counts of the user, the efforts and intensity of the user during exercise routines, the speed of performing the exercise by the user, and the halt times based on the automatic counting of the repetitions of the exercise and the time elapsed performing the exercise, when the exercise is being done by the user and providing instant feedback and appropriate exercise form guidance to the user.

8. The method of claim 1, wherein determining the plane of orientation of the user, determining the relative positions of the identified coordinates of the consecutive exercising poses of the user, the angles among the identified coordinates of the consecutive exercising poses of the user, and calculating the distance between at least two of the identified coordinates of the consecutive exercising poses of the user as a percentage of the predefined height further comprise of a predefined confidence interval to determine an acceptable error range.

9. The method of claim 1, wherein the automatically counting the repetitions and form guidance of the exercise using an exercise language further comprises information of one or more predefined exercises performed in sequence by the user.

10. The method of claim 9, wherein determining the plane of orientation of the consecutive exercising poses for the predefined exercises performed in sequence by the user corresponds to specific detected points of the user, and wherein the specific detected points of the user comprises horizontal, vertical, and angular plane to determine the plane of orientation of the user.

11. The method of claim 9, wherein said relative positions of the user for performing the predefined exercises comprises in front of, behind, above, below, the identified coordinates of the consecutive exercising poses of the user.

12. The method of claim 9, wherein processing the received consecutive exercising poses of the user for predefined exercises further comprises determining an end of a predefined exercise pose performed by the user.

13. A system for automatically counting a plurality of repetitions and providing form guidance of an exercise performed by a user in real time, the system comprising:
non-transitory computer readable storage media for storing computer program instructions defined by modules of the system;
processors communicatively coupled to the non-transitory computer readable storage media, the processors configured to execute the defined computer program instructions;
an exercise counting repetition and form guidance system for automatically counting a plurality of repetitions and providing form guidance of an exercise performed by a user in real time and executable by at least one of the processors configured to execute computer program instructions defined by modules of the exercise counting repetition and form guidance system, the modules of the exercise counting repetition and form guidance system comprising:
a capturing module for capturing one or more consecutive exercising poses of the exercise, performed by the user, in real time as a video stream;
a receiving module for receiving the captured consecutive exercising poses in real time, from the capturing module;
a language definition module for defining an exercise language comprising a finite set of instructions for automatically counting the repetitions of the exercise and providing form guidance to the user based on the received consecutive exercising poses;
an identification module for identifying a plurality of coordinates corresponding to a plurality of specific body joints of the user related to the consecutive exercising poses using the defined exercise language based on the received consecutive exercising poses of the user;
a normalization module for normalizing the captured consecutive exercising poses of the user based on the identified coordinates related to the consecutive exercising poses;
an analysis module for analyzing the identified coordinates associated with the normalized consecutive exercising poses of the user for determining a plane of orientation of the user;
a relative position determination module for determining one or more relative positions of the user based on the determination of the plane of orientation of the consecutive exercising poses of the user;
a distance calculation module for calculating a distance between at least two of the identified coordinates of the consecutive exercising poses of the user based on the determined plane of orientation and the determined relative positions of the identified coordinates of the consecutive exercising poses of the user as a percentage of the predefined height;
an angle calculation module for calculating a maximum angle and a minimum angle between three of the identified coordinates of the consecutive exercising poses of the user based on the calculated distance between at least two of the identified coordinates of the consecutive exercising poses of the user;
a processing module for processing the received consecutive exercising poses of the user by utilizing one or more stored instructions in the exercise counting repetition and form guidance system from the defined exercise language based on the calculated maximum angle and the minimum angle between three of the identified coordinates of the consecutive exercising poses of the user thereby automatically counting the repetitions of an exercise performed by the user; and
a form guidance module for providing an appropriate exercise form guidance to the user performing the exercise in real time based on the processed consecutive exercising poses of the user by verifying the exercise being performed by the user is in the appropriate form.

14. The system of claim 13, wherein the identification module for identifying the coordinates of the consecutive exercising poses of the user comprises a Convolutional Neural Network (CNN) and deep learning algorithms for detecting image and video objects, human pose estimation, and computer vision analysis.

15. The system of claim 13, where in the exercise language definition module automatically counts the repetitions and provides form guidance for a plurality of exercises using the deep learning methods and the finite set of instructions used in the exercise counting repetition and form guidance system.

16. The system of claim 13, wherein the normalization module further comprises a mapping module for mapping the height of the captured consecutive exercising poses of the user to a predefined standard height of the user, and selectively mapping the captured consecutive exercising poses of the user to their corresponding rotational equivalents.

17. The system of claim 13, wherein the capturing module eliminates the requirement for high bandwidth for streaming the captured consecutive exercising poses of the user for analysis aided by large computing environments.

18. The system of claim 13, wherein the analysis module, the relative position determination module, the distance calculation module, and the angle calculation module further comprises an error range determination module for determining an acceptable error range of a predefined confidence interval.

19. The system of claim 13, wherein the analysis module to determine the plane of orientation of the consecutive exercising poses for the predefined exercises performed in sequence by the user corresponds to specific detected points of the user.

20. A non-transitory computer readable storage medium having embodied thereon, computer program codes comprising instructions executable by at least one processor for automatically counting a plurality of repetitions of an exercise and providing form guidance performed by a user in real time, the computer program codes comprising:
a first computer program code for capturing one or more consecutive exercising poses of the exercise performed by the user, in real time as a video stream by a capturing module of an electronic device;
a second computer program code for receiving the captured consecutive exercising poses in real time, from the capturing module of the electronic device;
a third computer program code for defining an exercise language comprising a finite set of instructions for automatically counting the repetitions of the exercise and providing form guidance to the user based on the received consecutive exercising poses; a fourth computer program code for identifying a plurality of coordinates corresponding to a plurality of specific body joints of the user related to the consecutive exercising poses using the defined exercise language based on the received consecutive exercising poses of the user;
a fifth computer program code for normalizing the captured consecutive exercising poses of the user based on the identified coordinates related to the consecutive exercising poses;
a sixth computer program code for analyzing the identified coordinates associated with the normalized consecutive exercising poses of the user for determining a plane of orientation of the user;
a seventh computer program code for determining one or more relative positions of the user based on the determination of the plane of orientation of the consecutive exercising poses of the user, wherein the relative positions corresponds between and among the identified coordinates of the consecutive exercising poses of the user;
a eighth computer program code for calculating a distance between at least two of the identified coordinates of the consecutive exercising poses of the user based on the determined plane of orientation and the determined relative positions of the identified coordinates of the consecutive exercising poses of the user as a percentage of the predefined height;
an ninth computer program code for calculating a maximum angle and a minimum angle between three of the identified coordinates of the consecutive exercising poses of the user based on the calculated distance between at least two of the identified coordinates of the consecutive exercising poses of the user;
a tenth computer program code for processing the received consecutive exercising poses of the user by utilizing one or more stored instructions in the exercise counting repetition and form guidance system from the defined exercise language based on the calculated maximum angle and the minimum angle between three of the identified coordinates of the consecutive exercising poses of the user thereby automatically counting the repetitions of an exercise performed by the user; and
an eleventh computer program code for providing an appropriate exercise form guidance to the user performing the exercise in real time based on the processed consecutive exercising poses of the user by verifying the exercise being performed by the user is in the appropriate form.

21. The non-transitory computer readable storage medium of claim 20, wherein the fifth computer program code further comprises a twelfth computer program code for mapping a height of the captured consecutive exercising poses of the user to a predefined standard height of the user, and selectively mapping the captured consecutive exercising poses of the user to their corresponding rotational equivalents.

22. The non-transitory computer readable storage medium of claim 20, where in the defined exercise language in the exercise counting repetition and form guidance system automatically counts the repetitions and provides form guidance for a plurality of exercises using the deep learning methods and the finite set of instructions used in the exercise counting repetition and form guidance system.

23. The non-transitory computer readable storage medium of claim 20, further comprises an thirteenth computer program code for determining one or more calorie counts of the user, the efforts and intensity of the user during exercise routines, the speed of performing the exercise by the user, and the halt times based on the automatic counting of the repetitions of exercises and the time elapsed performing an exercise, when the exercise is being done by the user and providing instant feedback and appropriate exercise form guidance to the user.

24. The non-transitory computer readable storage medium of claim 21, further comprises a fourteenth computer program code for determining an acceptable error range for a predefined confidence interval of the exercise performed by the user.
